SEQUENCE LISTING (48 sequences)

SEQ ID NO: 1 - RNA, 22 nt, Rat
gcacugagaugggaguggugua

SEQ ID NO: 2 - RNA, 22 nt, Rat
acaguagucugcacauugguua

SEQ ID NO: 3 - RNA, 21 nt, Rat
aucacauugccagggauuacc

SEQ ID NO: 4 - RNA, 23 nt, Rat
uaauacugccugguaaugaugac

SEQ ID NO: 5 - RNA, 22 nt, Rat
cauugcacuugucucggucuga

SEQ ID NO: 6 - RNA, 23 nt, Rat
ugagguugguguacuguguguga

SEQ ID NO: 7 - RNA, 23 nt, Rat
agcagcauuguacagggcuauga

SEQ ID NO: 8 - RNA, 22 nt, Rat
ugagguaguagguuguaugguu

SEQ ID NO: 9 - RNA, 22 nt, Rat
ugagguaguagguuguauaguu

SEQ ID NO: 10 - RNA, 22 nt, Rat
agagguaguagguugcauaguu

SEQ ID NO: 11 - RNA, 21 nt, Rat
agaguaguagguugcauagua

SEQ ID NO: 12 - RNA, 22 nt, Rat
ccucccacacccaaggcuugca

SEQ ID NO: 13 - RNA, 23 nt, Rat
aacauucauugcugucggugggu

SEQ ID NO: 14 - RNA, 21 nt, Rat
aucacauugccagggauuacc

SEQ ID NO: 15 - RNA, 23 nt, Rat
caaagugcuguucgugcagguag

SEQ ID NO: 16 - RNA, 22 nt, Rat
uagcagcacguaaauauuggcg

SEQ ID NO: 17 - RNA, 23 nt, Rat
agcagcauuguacagggcuauga

SEQ ID NO: 18 - RNA, 21 nt, Rat
ucgaggagcucacagucuagu

SEQ ID NO: 19 - RNA, 22 nt, Rat
cacccguagaaccgaccuugcg

SEQ ID NO: 20 - RNA, 23 nt, Rat
uauggcuuuucauuccuauguga

SEQ ID NO: 21 - RNA, 22 nt, Rat
uaacacugucugguaacgaugu

SEQ ID NO: 22 - RNA, 23 nt, Rat
uaauacugccugguaaugaugac

SEQ ID NO: 23 - RNA, 22 nt, Rat
caucuuacugggcagcauugga

SEQ ID NO: 24 - RNA, 21 nt, Rat
uaauacugccggguaaugaug

SEQ ID NO: 25 - RNA, 22 nt, Rat
caacagcagucgaugggcuguc

SEQ ID NO: 26 - RNA, 22 nt, Rat
uagcuuaucagacugauguuga

SEQ ID NO: 27 - RNA, 22 nt, Rat
cgaaucauuauuugcugcucua

SEQ ID NO: 28 - RNA, 22 nt, Rat
uagcagcacaucaugguuuaca

SEQ ID NO: 29 - RNA, 22 nt, Rat
uagcagcacguaaauauuggcg

SEQ ID NO: 30 - RNA, 22 nt, Rat
uggagugugacaaugguguuug

SEQ ID NO: 31 - RNA, 22 nt, Rat
gugaaauguuuaggaccacuag

SEQ ID NO: 32 - RNA, 22 nt, Rat
uuuguucguucggcucgcguga

SEQ ID NO: 33 - protein, 1253 aa, Homo sapiens,
MKIITYFCIWAVAWAIPVPQSKPLERHVEKSMNLHLLARSNVSVQDELNASGTIKESGVLVHEGDRGRQENTQDGHKGEGNGSKWAEVGGKSFSTYSTLANEEGNIEGWNGDTGKAETYGHDGIHGKEENITANGIQGQVSIIDNAGATNRSNTNGNTDKNTQNGDVGDAGHNEDVAVVQEDGPQVAGSNNSTDNEDEIIENSCRNEGNTSEITPQINSKRNGTKEAEVTPGTGEDAGLDNSDGSPSGNGADEDEDEGSGDDEDEEAGNGKDSSNNSKGQEGQDHGKEDDHDSSIGQNSDSKEYYDPEGKEDPHNEVDGDKTSKSEENSAGIPEDNGSQRIEDTQKLNHRESKRVENRITKESETHAVGKSQDKGIEIKGPSSGNRNITKEVGKGNEGKEDKGQHGMILGKGNVKTQGEVVNIEGPGQKSEPGNKVGHSNTGSDSNSDGYDSYDFDDKSMQGDDPNSSDESNGNDDANSESDNNSSSRGDASYNSDESKDNGNGSDSKGAEDDDSDSTSDTNNSDSNGNGNNGNDDNDKSDSGKGKSDSSDSDSSDSSNSSDSSDSSDSDSSDSNSSSDSDSSDSDSSDSSDSDSSDSSNSSDSSDSSDSSDSSDSSDSSDSKSDSSKSESDSSDSDSKSDSSDSNSSDSSDNSDSSDSSNSSNSSDSSDSSDSSDSSSSSDSSSSSDSSNSSDSSDSSDSSNSSESSDSSDSSDSDSSDSSDSSNSNSSDSDSSNSSDSSDSSDSSDSSNSSDSSDSSDSSNSSDSSDSSDSSDSSDSSNSSDSNDSSNSSDSSDSSNSSDSSNSSDSSDSSDSSDSDSSNSSDSSNSSDSSDSSNSSDSSDSSDSSDSSDSDSSNRSDSSNSSDSSDSSDSSNSSDSSDSSDSSDSNESSNSSDSSDSSNSSDSDSSDSSNSSDSSDSSNSSDSSESSNSSDNSNSSDSSNSSDSSDSSDSSNSSDSSNSGDSSNSSDSSDSNSSDSSDSSNSSDSSDSSDSSDSSDSSDSSNSSDSSDSSDSSDSSNSSDSSNSSDSSDSSDSSDSSDSSDSSNSSDSSDSSDSSDSSDSSGSSDSSDSSDSSDSSDSSDSSDSSDSSDSSESSDSSDSSDSSDSSDSSDSSDSSDSSDSSDSSDSSNSSDSSDSSDSSDSSDSSDSSDSSDSSDSSDSSDSSDSSDSSDSSDSSDSSDSNESSDSSDSSDSSDSSNSSDSSDSSDSSDSTSDSNDESDSQSKSGNGNNNGSDSDSDSEGSDSNHSTSDD

SEQ ID NO: 34 - protein, 349 aa, Homo sapiens [sequence omitted]

SEQ ID NO: 35 - protein, 198 aa, Homo sapiens
MASGNARIGKPAPDFKATAVVDGAFKEVKLSDYKGKYVVLFFYPLDFTFVCPTEIIAFSNRAEDFRKLGCEVLGVSVDSQFTHLAWINTPRKEGGLGPLNIPLLADVTRRLSEDYGVLKTDEGIAYRGLFIIDGKGVLRQITVNDLPVGRSVDEALRLVQAFQYTDEHGEVCPAGWKPGSDTIKPNVDDSKEYFSKHN

SEQ ID NO: 36 - protein, 279 aa, Homo sapiens
MKIIILLGFLGATLSAPLIPQRLMSASNSNELLLNLNNGQLLPLQLQGPLNSWIPPFSGILQQQQQAQIPGLSQFSLSALDQFAGLLPNQIPLTGEASFAQGAQAGQVDPLQLQTPPQTQPGPSHVMPYVFSFKMPQEQGQMFQYYPVYMVLPWEQPQQTVPRSPQQTRQQQYEEQIPFYAQFGYIPQLAEPAISGGQQQLAFDPQLGTAPEIAVMSTGEEIPYLQKEAINFRHDSAGVFMPSTSPKPSTTNVFTSAVDQTITPELPEEKDKTDSLREP

SEQ ID NO: 37 - protein, 216 aa, Rattus norvegicus
MLYSKINNCKFDEFFSAGCAPGSPRNSSSLCALCIGSEKGTGKECVPNSNERYYGYTGAFRCLVEKGDVAFVKDQTVIQNTDGNNNEAWAKNMKKENFEVLCKDGTRKPVTDAENCHLPEPNHAVVSRKDKATCVEKILNKQQDDFGKSVTDCTSNFCLFQSNSKDLLFRDDTKCLASIAKKTYDSYLGDDYVRAMTNLRQCSTSKLLEACTFHKP

SEQ ID NO: 38 - protein, 167 aa, Homo sapiens
MGNCVKYPLRNLSRKDRSLRPEEIEELREAFREFDKDKDGYINCRDLGNCMRTMGYMPTEMELIELSQQINMNLGGHVDFDDFVELMGPKLLAETADMIGVKELRDAFREFDTNGDGEISTSELREAMRKLLGHQVGHRDIEEIIRDVDLNGDGRVDFEEFVRMMSR

SEQ ID NO: 39 - protein, 308 aa, Homo sapiens
MWKRWLALALALVAVAWVRAEEELRSKSKICANVFCGAGRECAVTEKGEPTCLCIEQCKPHKRPVCGSNGKTYLNHCELHRDACLTGSKIQVDYDGHCKEKKSVSPSASPVVCYQSNRDELRRRIIQWLEAEIIPDGWFSKGSNYSEILDKYFKNFDNGDSRLDSSEFLKFVEQNETAINITTYPDQENNKLLRGLCVDALIELSDENADWKLSFQEFLKCLNPSFNPPEKKCALEDETYADGAETEVDCNRCVCACGNWVCTAMTCDGKNQKGAQTQTEEEMTRYVQELQKHQETAEKTKRVSTKEI

SEQ ID NO: 40 - protein, 166 aa, Homo sapiens
MASGVAVSDGVIKVFNDMKVRKSSTPEEVKKRKKAVLFCLSEDKKNIILEEGKEILVGDVGQTVDDPYATFVKMLPDKDCRYALYDATYETKESKKEDLVFIFWAPESAPLKSKMIYASSKDAIKKKLTGIKHELQANCYEEVKDRCTLAEKLGGSAVISLEGKPL

SEQ ID NO: 41 - protein, 339 aa, Homo sapiens
MSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 42 - protein, 335 aa, Homo sapiens
MPRPRLLAALCGALLCAPSLLVALDICSKNPCHNGGLCEEISQEVRGDVFPSYTCTCLKGYAGNHCETKCVEPLGLENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQIFPGNWDNHSHKKNLFETPILARYVRILPVAWHNRIALRLELLGC

SEQ ID NO: 43 - protein, 418 aa, Homo sapiens
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 44 - protein, 1299 aa, Homo sapiens
MSLQEMFRFPMGLLLGSVLLVASAPATLEPPGCSNKEQQVTVSHTYKIDVPKSALVQVDADPQPLSDDGASLLALGEAREEQNIIFRHNIRLQTPQKDCELAGSVQDLLARVKKLEEEMVEMKEQCSAQRCCQGVTDLSRHCSGHGTFSLETCSCHCEEGREGPACERLACPGACSGHGRCVDGRCLCHEPYVGADCGYPACPENCSGHGECVRGVCQCHEDFMSEDCSEKRCPGDCSGHGFCDTGECYCEEGFTGLDCAQVVTPQGLQLLKNTEDSLLVSWEPSSQVDHYLLSYYPLGKELSGKQIQVPKEQHSYEILGLLPGTKYIVTLRNVKNEVSSSPQHLLATTDLAVLGTAWVTDETENSLDVEWENPSTEVDYYKLRYGPMTGQEVAEVTVPKSSDPKSRYDITGLHPGTEYKITVVPMRGELEGKPILLNGRTEIDSPTNVVTDRVTEDTATVSWDPVQAVIDKYVVRYTSADGDTKEMAVHKDESSTVLTGLKPGEAYKVYVWAERGNQGSKKADTNALTEIDSPANLVTDRVTENTATISWDPVQATIDKYVVRYTSADDQETREVLVGKEQSSTVLTGLRPGVEYTVHVWAQKGDRESKKADTNAPTDIDSPKNLVTDRVTENMATVSWDPVQAAIDKYVVRYTSAGGETREVPVGKEQSSTVLTGLRPGMEYMVHVWAQKGDQESKKADTKAQTDIDSPQNLVTDRVTENMATVSWDPVRATIDRYVVRYTSAKDGETREVPVGKEQSSTVLTGLRPGVEYTVHVWAQKGAQESKKADTKAQTDIDSPQNLVTDWVTENTATVSWDPVQATIDRYVVHYTSANGETREVPVGKEQSSTVLTGLRPGMEYTVHVWAQKGNQESKKADTKAQTEIDGPKNLVTDWVTENMATVSWDPVQATIDKYMVRYTSADGETREVPVGKEHSSTVLTGLRPGMEYMVHVWAQKGAQESKKADTKAQTELDPPRNLRPSAVTQSGGILTWTPPSAQIHGYILTYQFPDGTVKEMQLGREDQRFALQGLEQGATYPVSLVAFKGGRRSRNVSTTLSTVGARFPHPSDCSQVQQNSNAASGLYTIYLHGDASRPLQVYCDMETDGGGWIVFQRRNTGQLDFFKRWRSYVEGFGDPMKEFWLGLDKLHNLTTGTPARYEVRVDLQTANESAYAIYDFFQVASSKERYKLTVGKYRGTAGDALTYHNGWKFTTFDRDNDIALSNCALTHHGGWWYKNCHLANPNGRYGETKHSEGVNWEPWKGHEFSIPYVELKIRPHGYSREPVLGRKKRTLRGRLRTF

SEQ ID NO: 45 - protein, 590 aa, Homo sapiens
MSRQSSVSFRSGGSRSFSTASAITPSVSRTSFTSVSRSGGGGGGGFGRVSLAGACGVGGYGSRSLYNLGGSKRISISTSGGSFRNRFGAGAGGGYGFGGGAGSGFGFGGGAGGGFGLGGGAGFGGGFGGPGFPVCPPGGIQEVTVNQSLLTPLNLQIDPSIQRVRTEEREQIKTLNNKFASFIDKVRFLEQQNKVLDTKWTLLQEQGTKTVRQNLEPLFEQYINNLRRQLDSIVGERGRLDSELRNMQDLVEDFKNKYEDEINKRTTAENEFVMLKKDVDAAYMNKVELEAKVDALMDEINFMKMFFDAELSQMQTHVSDTSVVLSMDNNRNLDLDSIIAEVKAQYEEIANRSRTEAESWYQTKYEELQQTAGRHGDDLRNTKHEISEMNRMIQRLRAEIDNVKKQCANLQNAIADAEQRGELALKDARNKLAELEEALQKAKQDMARLLREYQELMNTKLALDVEIATYRKLLEGEECRLSGEGVGPVNISVVTSSVSSGYGSGSGYGGGLGGGLGGGLGGGLAGGSSGSYYSSSSGGVGLGGGLSVGGSGFSASSGRGLGVGFGSGGGSSSSVKFVSTTSSSRKSFKS

SEQ ID NO: 46 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 47 - protein, 100 aa, Homo sapiens
MRALTLLALLALAALCIAGQAGAKPSGAESSKGAAFVSKQEGSEVVKRPRRYLYQWLGAPVPYPDPLEPRREVCELNPDCDELADHIGFQEAYRRFYGPV

SEQ ID NO: 48 - protein, 524 aa, Homo sapiens
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

(19) United States
(12) Patent Application Publication
(10) Pub. No.: US 2016/0120805 A1
(43) Pub. Date: May 5, 2016

(54) EXOSOMES FOR OROFACIAL DIAGNOSTICS AND THERAPEUTICS

(71) Applicant: THE TRUSTEES OF COLUMBIA UNIVERSITY IN THE CITY OF NEW YORK, New York, NY (US)

(72) Inventors: Jeremy J. Mao, Closter, NJ (US); Ying Wan, New York, NY (US); Nan Jiang, New York, NY (US); Mo Chen, Astoria, NY (US)

(73) Assignee: The Trustees of Columbia University in the City of New York, New York, NY (US)

(21) Appl. No.: 14/896,017

(22) PCT Filed: Jun. 5, 2014
(86) PCT No.: PCT/US14/41189

§ 371 Date: Dec. 4, 2015

Related U.S. Application Data
(60) Provisional application No. 61/976,988, filed on Apr. 8, 2014.
(60) Provisional application No. 61/831,602, filed on Jun. 5, 2013.

(51) Int. Cl. A61K 9/127; A61K 38/17; A61K 31/7105

(52) U.S. Cl. CPC A61K 9/127; A61K 31/7105; A61K 38/1709

(57) ABSTRACT

Provided are methods of treating a subject with a composition comprising an exosome or a polypeptide, RNA, or miRNA contained therein or identified or isolated therefrom. Also provided are methods of promoting dentinogenesis, amelogenesis, or osteogenesis. Also provided are compositions comprising an exosome or a polypeptide, RNA, or miRNA contained therein or identified or isolated therefrom.

### CROSS REFERENCE TO RELATED APPLICATIONS

The present application claims the benefit of priority to U.S. Provisional Application Ser. No. 61/976,988 filed 8 Apr. 2014, and U.S. Provisional Application Ser. No. 61/831,602 filed 5 Jun. 2013, each of which are incorporated herein by reference in its entirety.

### STATEMENT REGARDING FEDERALLY SPONSORED RESEARCH OR DEVELOPMENT

This invention was made with government support under grant numbers 5RC2DE020767, RC2DE020767, R01EB009663, and R01DE023112 awarded by the National Institute of Dental and Craniofacial Research of the National Institutes of Health. The government has certain rights in the invention.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### BACKGROUND OF THE INVENTION

Exosomes are vesicles of endocytic origin released by many cells. Exosomes are small vesicular structures averaging 40-120 nm (generally <200 nm) in diameter and are distinguished by their formation within cellular endosomal compartments known as multivesicular bodies (MVBs). Exosomes can contain proteins, peptides, and RNA.

Exosomes were initially discovered in 1983, and are presently thought to play a role in intercellular communication. It is generally understood that exosomes can be secreted by most cell types and can carry molecular messages through combinations of proteins, mRNA, or miRNA specific to the cellular source.

Odontogenesis, or tooth development, involves an intricate sequence of reciprocal signaling between dental epithelial and dental mesenchymal cells that is only partly understood. In a study by Theslef et al., to elucidate the mechanism for signal transmission in early odontogenesis, it was demonstrated that interposition of a nucleopore filter with pore size >200-nm would permit normal cytodifferentiation of odontoblasts and ameloblasts whereas pore size of 100-nm prevented cytodifferentiation (Theslef et al. 1977 Dev Biol 58, 197-203). Theslef concluded from the findings that juxtacrine (contact-dependent) signaling must be the sole means of intercellular communication because diffusible signals would have traversed the smaller pores had they been present.

Exosomes are understood to play a role in intercellular communication. Exosomes have an evolutionarily conserved set of proteins including CD81, CD63, CD9, Alix, and Tsg101.

### SUMMARY OF THE INVENTION

Among the various aspects of the present disclosure is the provision of a method of treating a subject for a mineralization injury, disease or disorder. Another aspect is the provision of a method of promoting dentinogenesis, amelogenesis, or odontogenesis in a subject.

In some embodiments, the method includes administering to a subject in need thereof a composition comprising (i) an exosome or (ii) one or more of a polypeptide, mRNA, or miRNA associated with or derived from the exosome. In some embodiments, the method includes contacting the composition and a dental cell, such as a mesenchyme, epithelium cell, or a mesoderm cell.

In some embodiments, such administration results in one or more of increased expression of dentin sialophosphoprotein (DSPP) expression, increased expression of osteocalcin (OCN) expression, increased expression of alkaline phosphatase, promotion of promote calcium deposition, promotion of dentinogenesis, promotion of amelogenesis, or promotion of odontogenesis.

In some embodiments, the exosome comprises an epithelium-derived exosome; mesenchyme-derived exosome; or a mesoderm-derived exosome. In some embodiments, the method includes isolating the exosome from an epithelium cell, a mesenchyme cell, or a mesoderm cell. In some embodiments, the method further includes isolating the exosome from a tooth epithelium cell, a tooth mesenchyme cell, or a tooth mesoderm cell. In some embodiments, the exosome has a particle size of about 80 to about 120 nm. In some embodiments, a plurality of epithelium-derived exosomes have an average particle size of about 95 nm to about 105 nm. In some embodiments, a plurality of mesenchyme-derived exosomes have an average particle size of about 110 nm to about 120 nm.

In some embodiments, the composition comprises an epithelium-derived exosome, the exosome comprising one or more of (a) a miRNA comprising a nucleic acid sequence selected from the group consisting of SEQ ID NO: 1 (mo-miR-674-5p), SEQ ID NO: 2 (rno-miR-199a-3p), SEQ ID NO: 3 (rno-miR-23b-3p), SEQ ID NO: 4 (rno-miR-200b-3p£°), SEQ ID NO: 5 (rno-miR-25-3p), SEQ ID NO: 6 (rno-miR-672-5p£°), and SEQ ID NO: 7 (rno-miR-103-3p£°), or a nucleic acid sequence having at least about 90% sequence identity thereto and retaining an activity associated with the miRNA; or (b) a polypeptide comprising an amino acid sequence of SEQ ID NO: 34 (CTGF), SEQ ID NO: 35 (peroxiredoxin-2), SEQ ID NO: 36 (odontogenic ameloblast-associated protein precursor), SEQ ID NO: 37 (hemiferrin, transferrin-like protein), SEQ ID NO: 38 (CaBP1), SEQ ID NO: 39 (follistatin-related protein 1 precursor), and SEQ ID NO: SEQ ID NO: 40 (cofilin-1), or an amino acid sequence having at least about 90% sequence identity thereto and retaining an activity associated with the polypeptide.

In some embodiments, the composition comprises one or more of: (a) a miRNA comprising a nucleic acid sequence selected from the group consisting of SEQ ID NO: 1 (rno-miR-674-5p), SEQ ID NO: 2 (rno-miR-199a-3p), SEQ ID NO: 3 (rno-miR-23b-3p), SEQ ID NO: 4 (rno-miR-200b-3p£°), SEQ ID NO: 5 (rno-miR-25-3p), SEQ ID NO: 6 (rno-miR-672-5p£°), and SEQ ID NO: 7 (rno-miR-103-3p£°), or a nucleic acid sequence having at least about 90% sequence identity thereto and retaining an activity associated with the miRNA; (b) a polypeptide comprising an amino acid sequence of SEQ ID NO: 34 (CTGF), SEQ ID NO: 35 (peroxiredoxin-2), SEQ ID NO: 36 (odontogenic ameloblast-associated protein precursor), SEQ ID NO: 37 (hemiferrin, transferrin-like protein), SEQ ID NO: 38 (CaBP1), SEQ ID NO: 39 (follistatin-related protein 1 precursor), and SEQ ID NO: SEQ ID NO: 40 (cofilin-1), or an amino acid sequence having at least about 90% sequence identity thereto and retaining an activity associated with the polypeptide; or (c) a vector comprising a transcribable nucleic acid molecule encoding the miRNA or the polypeptide operably linked to a promoter.

In some embodiments, the composition promotes amelogenesis.

In some embodiments, the composition comprises an mesenchyme-derived exosome, the exosome comprising one or more of: (a) a miRNA comprising a nucleic acid sequence selected from the group consisting of SEQ ID NO: 8 (rno-let-7c-5p£°), SEQ ID NO: 9 (rno-let-7a-5p£°), SEQ ID NO: 10 (rno-let-7d-5p£°), SEQ ID NO: 11 (rno-miR-352£°), SEQ ID NO: 12 (rno-miR-532-3p£°), SEQ ID NO: 13 (rno-miR-181b-5p£°), SEQ ID NO: 14 (rno-miR-23b-3p£°), SEQ ID NO: 15 (rno-miR-93-5p£°), SEQ ID NO: 16 (rno-miR-16-5p£°), SEQ ID NO: 17 (rno-miR-103-3p£°), SEQ ID NO: 18 (rno-miR-151-5p£°), and SEQ ID NO: 19 (rno-miR-99b-5p£°), or a nucleic acid sequence having at least about 90% sequence identity thereto and retaining an activity associated with the miRNA; or (b) a polypeptide comprising an amino acid sequence of SEQ ID NO: 41 (annexin II), SEQ ID NO: 42 (lactadherin isoform b precursor), SEQ ID NO: 43 (pigment epithelium-derived factor precursor), SEQ ID NO: 44 (tenascin-N precursor), SEQ ID NO: 45 (keratin, type II cytoskeletal 5), and SEQ ID NO: 46 (periostin isoform 1 precursor), or an amino acid sequence having at least about 90% sequence identity thereto and retaining an activity associated with the polypeptide.

In some embodiments, the composition comprises one or more of: (a) a miRNA comprising a nucleic acid sequence selected from the group consisting of SEQ ID NO: 8 (rno-let-7c-5p£°), SEQ ID NO: 9 (rno-let-7a-5p£°), SEQ ID NO: 10 (rno-let-7d-5p£°), SEQ ID NO: 11 (rno-miR-352£°), SEQ ID NO: 12 (rno-miR-532-3p£°), SEQ ID NO: 13 (rno-miR-181b-5p£°), SEQ ID NO: 14 (rno-miR-23b-3p£°), SEQ ID NO: 15 (rno-miR-93-5p£°), SEQ ID NO: 16 (rno-miR-16-5p£°), SEQ ID NO: 17 (rno-miR-103-3p£°), SEQ ID NO: 18 (rno-miR-151-5p£°), and SEQ ID NO: 19 (rno-miR-99b-5p£°), or a nucleic acid sequence having at least about 90% sequence identity thereto and retaining an activity associated with the miRNA; (b) a polypeptide comprising an amino acid sequence of SEQ ID NO: 41 (annexin II), SEQ ID NO: 42 (lactadherin isoform b precursor), SEQ ID NO: 43 (pigment epithelium-derived factor precursor), SEQ ID NO: 44 (tenascin-N precursor), SEQ ID NO: 45 (keratin, type II cytoskeletal 5), and SEQ ID NO: 46 (periostin isoform 1 precursor), or an amino acid sequence having at least about 90% sequence identity thereto and retaining an activity associated with the polypeptide; or (c) a vector comprising a transcribable nucleic acid molecule encoding the miRNA or the polypeptide operably linked to a promoter.

In some embodiments, the composition promotes odontogenesis.

In some embodiments, the exosome comprises one or more of: a miRNA comprising a nucleic acid sequence selected from the group consisting of SEQ ID NO: 20 (rno-miR-135b-5p£°), SEQ ID NO: 21 (rno-miR-200a-3p£°), SEQ ID NO: 22 (rno-miR-200b-3p£°), SEQ ID NO: 23 (rno-miR-200b-5p£°), SEQ ID NO: 24 (rno-miR-200c-3p£°), SEQ ID NO: 25 (rno-miR-21-3p£°), SEQ ID NO: 26) (rno-miR-21-3p£°), SEQ ID NO: 27 (rno-miR-15b-3p£°), SEQ ID NO: 28 (rno-miR-15b-5p£°), SEQ ID NO: 29 (rno-miR-16-5p£°), SEQ ID NO: 30 (rno-miR-122-5p£°), SEQ ID NO: 31 (rno-miR-203a-3p£°), and SEQ ID NO: 32 (rno-miR-375-3p£°), or a nucleic acid sequence having at least about 90% sequence identity thereto and retaining an activity associated with the miRNA.

In some embodiments, the composition comprises: (a) one or more of a miRNA comprising a nucleic acid sequence selected from the group consisting of SEQ ID NO: 20 (rno-miR-135b-5p£°), SEQ ID NO: 21 (rno-miR-200a-3p£°), SEQ ID NO: 22 (rno-miR-200b-3p£°), SEQ ID NO: 23 (rno-miR-200b-5p£°), SEQ ID NO: 24 (rno-miR-200c-3p£°), SEQ ID NO: 25 (rno-miR-21-3p£°), SEQ ID NO: 26 (rno-miR-21-3p£°), SEQ ID NO: 27 (rno-miR-15b-3p£°), SEQ ID NO: 28 (rno-miR-15b-5p£°), SEQ ID NO: 29 (rno-miR-16-5p£°), SEQ ID NO: 30 (rno-miR-122-5p£°), SEQ ID NO: 31 (rno-miR-203a-3p£°), and SEQ ID NO: 32 (rno-miR-375-3p£°), or a nucleic acid sequence having at least about 90% sequence identity thereto and retaining an activity associated with the miRNA; or (b) a vector comprising a transcribable nucleic acid molecule encoding the miRNA operably linked to a promoter.

In some embodiments, the subject is a mammal. In some embodiments, the subject is a human.

In some embodiments, the mineralization injury, disease or disorder is selected from the group consisting of bone fracture, tooth extraction sockets, periodontal defects, non-unions, dental and orthopedic implant integration, and bony augmentation in reconstructive or plastic procedures.

Another aspect provides a composition for treating a mineralization injury, disease or disorder or for promoting dentinogenesis, amelogenesis, or odontogenesis.

In some embodiments, the composition includes an epithelium-derived exosome, a mesenchyme-derived exosome, or a mesoderm-derived exosome.

In some embodiments, the composition includes (a) an epithelium-derived exosome, a mesenchyme-derived exosome, or a mesoderm-derived exosome and (b) one or more of the following: (i) a miRNA comprising a nucleic acid sequence selected from the group consisting of SEQ ID NO: 1 (rno-miR-674-5p), SEQ ID NO: 2 (rno-miR-199a-3p), SEQ ID NO: 3 (rno-miR-23b-3p), SEQ ID NO: 4 (rno-miR-200b-3p£°), SEQ ID NO: 5 (rno-miR-25-3p), SEQ ID NO: 6 (rno-miR-672-5p£°), and SEQ ID NO: 7 (rno-miR-103-3p£°), or a nucleic acid sequence having at least about 90% sequence identity thereto and retaining an activity associated with the miRNA; (ii) a polypeptide comprising an amino acid sequence of SEQ ID NO: 34 (CTGF), SEQ ID NO: 35 (peroxiredoxin-2), SEQ ID NO: 36 (odontogenic ameloblast-associated protein precursor), SEQ ID NO: 37 (hemiferrin, transferrin-like protein), SEQ ID NO: 38 (CaBP1), SEQ ID NO: 39 (follistatin-related protein 1 precursor), and SEQ ID NO: SEQ ID NO: 40 (cofilin-1), or an amino acid sequence having at least about 90% sequence identity thereto and retaining an activity associated with the polypeptide; (iii) a miRNA comprising a nucleic acid sequence selected from the group consisting of SEQ ID NO: 1 (rno-miR-674-5p), SEQ ID NO: 2 (rno-miR-199a-3p), SEQ ID NO: 3 (rno-miR-23b-3p), SEQ ID NO: 4 (rno-miR-200b-3p£°), SEQ ID NO: 5 (rno-miR-25-3p), SEQ ID NO: 6 (rno-miR-672-5p£°), and SEQ ID NO: 7 (rno-miR-103-3p£°), or a nucleic acid sequence having at least about 90% sequence identity thereto and retaining an activity associated with the miRNA; (iv) a polypeptide comprising an amino acid sequence of SEQ ID NO: 34 (CTGF), SEQ ID NO: 35 (peroxiredoxin-2), SEQ ID NO: 36 (odontogenic ameloblast-associated protein precursor), SEQ ID NO: 37 (hemiferrin, transferrin-like protein), SEQ ID NO: 38 (CaBP1), SEQ ID NO: 39 (follistatin-related protein 1 precursor), and SEQ ID NO: SEQ ID NO: 40 (cofilin-1), or an amino acid sequence having at least about 90% sequence identity thereto and retaining an activity associated with the polypeptide; or (v) a vector comprising a transcribable nucleic acid molecule encoding the miRNA or the polypeptide operably linked to a promoter.

In some embodiments, wherein the (b) component is independently present in the composition. In some embodiments, wherein the (b) component is contained within the exosome.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-J′ is a series of diagrams, images, and illustrations depicting exosome isolation and characterization of secreted vesicles from dental epithelium and mesenchyme cells in 5/6-day-old SD rat incisors. Dental epithelium (FIG. 1A) was dissected from dental mesenchyme (FIG. 1C) under dissection microscope. Stem/progenitor cells from dental epithelium (FIG. 1B) and mesenchyme (FIG. 1D) were cultured in exosome-free medium at Day 3. (E) FIG. 1E-F shows the average diameter of particles (nm) as a function of concentration (particles/nm) per Nanoparticle Tracking Analysis (NTA), where average diameter of particles purified from dental epithelium was 100 nm (FIG. 1E) and from mesenchyme was 116 nm per (FIG. 1F), falling within the accepted range of exosome size. FIG. 1G shows anti-CD63 (a putative exosomal biomarker) antibody and anti-GM-130 antibody probe reactivity to total proteins extracted from the particles. FIG. 1H-J′ show immunofluorescence of 5-day-old rat incisor apical end, where FIG. 1H′, I′, J′ shows higher magnification of rectangular areas in FIG. 1H, I, J respectively. FIG. 1H-H′ show CD63 probe; FIG. 1I-I′ shows Dapi probe; and FIG. 1J-J′ shows CD63 and Dapi probes. Both epithelium (e) and adjacent mesenchyme (m) expressed CD63 especially in the cervical loop (FIG. 1H, I, J).

FIG. 2A-H′ is a series of images showing Cy™3 labeled siRNA electroporated exosome tracking. FIG. 2A-D show dental epithelium stem/progenitor cells incubated with mesenchymal exosomes for 24 h, where approximately 40% epithelium cells were positive for labeled exosome. FIG. 2A′-D′ show no positive Cy™3 was present in control epithelium cells. FIG. 2E-H show dental mesenchyme stem/progenitor cells incubated with epithelial exosomes for 24 h, where approximately 34% mesenchyme cells were positively labeled with exosomes. FIG. 2E′-H′ show no positive Cy™3 was present in control mesenchyme cells.

FIG. 3 is an image of silver stained gel showing the protein content of epithelial exosome, mesenchymal exosome, and the protein marker. Proteins extracted from epithelium and mesenchyme exosomes were loaded onto a 4-12% SDS-PAGE gel, followed by silver staining. The rectangle areas in the gel image represent the fraction analyzed by high-resolution mass spectrometry. Exemplary identified proteins from the epithelial exosome region are identified in TABLE 1. Exemplary identified proteins from the mesenchyme exosome region are identified in TABLE 2.

FIG. 4A-F is a series of bar graphs showing gene expression in exosome co-cultures. FIG. 4A is a series of bar graphs showing gene expression of Ameloblastin, Amelogenin, and Alp in co-cultures of Epi with Mexo @D4. FIG. 4B is a series of bar graphs showing gene expression of Ameloblastin, Amelogenin, and Alp in co-cultures of Epi with Mexo @D6. FIG. 4C is a series of bar graphs showing gene expression of Ameloblastin, Amelogenin, and Alp in co-cultures of Epi with Mexo @D9. FIG. 4D is a series of bar graphs showing gene expression of Alp, Dspp (SEQ ID NO: 33), Oc, and RunX2 in co-cultures of Mes with Eexo @D7. FIG. 4E is a series of bar graphs showing gene expression of Alp, Dspp (SEQ ID NO: 33), Oc, and RunX2 in co-cultures of Mes with Eexo @D14. FIG. 4E is a series of bar graphs showing gene expression of Alp, Dspp (SEQ ID NO: 33), Oc, and RunX2 in co-cultures of Mes with Eexo @D24.

FIG. 5A-B is a series of bar graphs showing RT-PCR data from the differentiation analysis experiments. FIG. 5A shows relative expressions of alkaline phosphatase (Alpl), dentin sialophosphoprotein (Dspp), osteocalcin (OC), and Runt-related transcription factor 2 (RunX2) are shown for mesenchymal cells originating from dental pulp exposed to varying concentrations of dental epithelial exosomes and at multiple timepoints. There is striking upregulation of Dspp of greater than 20-fold compared to control. FIG. 5B shows relative expression of ameloblastin (Ambn), amelogenin (Amgn), and alkaline phosphatase (Alpl) are shown for dental epithelial cells (FIG. 5B) exposed to dental mesenchymal exosomes.

FIG. 6A-D is a series of plots showing miRNAs encapsulated by exosomes. FIG. 6A-B show microRNA profiles of epithelial exosomes and their parental cells (dental epithelium stem/progenitor cells) analyzed using microRNA array by miRCURY LNA™ and EXIQON. TABLE 3 shows arbitrarily selected microRNAs in epithelium-derived exosomes. FIG. 6C-D shows microRNA profiles of epithelial exosomes and their parental cells (dental mesenchyme stem/progenitor cells) analyzed using microRNA array by miRCURY LNA™ and EXIQON. TABLE 4 shows arbitrarily selected microRNAs in mesenchyme-derived exosomes.

FIG. 7 is a series of cartoons, bar graphs, and gel images showing that dental mesenchyme exosomes promote differentiation towards amelogenesis. FIG. 7A depicts dental epithelium stem/progenitor cells incubated with exosomes secreted by dental mesenchyme stem/progenitor cells for 4 days. Dental mesenchyme exosomes induced upregulation of ameloblastin (AMBN) and amelogenin (AMELX) at gene level (FIG. 7B) and protein level (FIG. 7C), key markers for amelogenesis. Dental epithelium stem/progenitor cells were treated with dental mesenchyme exosomes with the presence of ascorbic acid (AA), and showed upregulation of basement membrane components, such as Col4a, Itga, Iam and Nid, at gene level (FIG. 7D) and protein level (FIG. 7E), suggesting that dental mesenchyme transmits amelogenic signal to epithelium via exosomes.

FIG. 8 is a series of bar graphs, plate images, and gel images showing that dental epithelium exosomes promote differentiation towards odontogenesis. FIG. 8A-B shows change of expression levels of DSPP, OC, and RUNX2 in dental mesenchyme stem/progenitor cells incubated with exosomes secreted by dental epithelium stem/progenitor cells for 14 (2 w) and 21 days (3 w). In FIG. 8A-B, dental epithelial exosomes induced robust upregulation of Dspp. FIG. 8C is an image of a Western Blot showing an increase of DSP and OCN. FIG. 8D is a series of images showing dental epithelial exosomes induced increase expression of alkaline phosphatase expression when dental mesenchyme stem/progenitor cells were cultured in osteogenesis medium for one week. FIG. 8E is a bar graph showing quantification of alkaline phosphatase expression for 1 week and 2 weeks. FIG. 8F is a series of images of Alizarin Red staining that shows epithelium exosomes promote calcium deposition and quantified in FIG. 8G. FIG. 8H is a series of bar graphs showing RT-PCR results, where dental epithelial exosomes induced robust upregulation of Dspp, a key transcriptional factors for odontogenesis. These data indicate that dental epithelium transmits odontogenic signal to mesenchyme via exosomes.

FIG. 9 is a series of images, line and scatter plots, and bar graphs showing that exosome deficiency resulted in the delay of tooth development. E16.5 epithelium and mesenchyme tissue (FIG. 9A) were reconstituted (FIG. 9B) under dissection microscope. The reconstituted organ were cultured for 12 days (FIG. 9C-D). Histology results showed robust dentin formation and cell polarization (FIG. 9E-G). Exosome inhibitor GW4869 didn't affect the cell proliferation significantly (FIG. 9H). GW4869 1.0 uM and 10.0 uM decreased the exosome secretion measured by protein concentration (FIG. 9I). In FIG. 9J-K. organ culture showed dentin formation at day 10 in the control group, where FIG. 9J is bright field and FIG. 9K is histological section followed by H&E staining. In FIG. 9L-M shows reconstitution of dental epithelium and mesenchyme tissues in the presence of GW4869 10 uM. No dentin formed as shown in FIG. 9M.

FIG. 10 is a series of images and bar graphs showing exosome deficiency resulted in the delay of tooth development as Rab27A and Rab27B were knocked down. In FIG. 10A, dental mesenchyme cells were transfected with Rab27A and Rab27B siRNA. The efficiency of knock down were measured by western blot and exosome secretion measured by protein concentration (FIG. 10B). In FIG. 10C-E, organ culture showed basement formation at day 4 in the control group, where FIG. 10C is bright field and FIG. 10D is histological section followed by H&E staining. FIG. 10E is immunofluorescence staining for type IV collagen. In FIG. 10E-H, reconstitution of dental epithelium and mesenchyme tissues as Rab27A and Rab27B were knocked down. Reduced collagen IV could be detected in FIG. 10H.

FIG. 11 is a series of bar graphs showing exosomes participate in the BMP and Wnt signaling pathway. FIG. 11A-B shows relative luciferase activities driven by epithelium stem/progenitor derived exosomes. Assays were performed in dental mesenchyme stem/progenitor cells harboring either 12XSBE (BMP) or Topflash (Wnt) expression vector. FIG. 11C shows selected miRNAs −ΔCT value in epithelium cells (EC), epithelium-derived exosomes (Eexo) and mesenchyme cells (MC), which are related with WNT/beta-catenin signaling pathway.

### DETAILED DESCRIPTION OF THE INVENTION

The present disclosure is based, at least in part, on the discovery that exosomes are contributors in epithelial-mesenchymal dialogue during odontogenesis. Accordingly, exosomes secreted by epithelium cells, mesenchyme cells, or mesoderm cells contain specific polypeptides or RNA that can act as diagnostic and therapeutic agents in a broad range of diseases and trauma.

Studies described herein show that dental epithelial or mesenchymal cells secrete exosomes as vehicles for intercellular signaling during odontogenesis. The following provides a brief over view of studies described in more depth in the example. Stem/progenitor cells were isolated by microdissection from the epithelium and mesenchyme in postnatal 4-5 day-old rodent incisors and cultured separately in exosome-free medium for 1 wk. Vesicles from culture medium were harvested by ultracentrifuge. By nanoparticle tracking analysis (NTA), epithelium- or mesenchyme-derived vesicles were separately isolated and verified to be in the range of 80-120 nm. Western blotting confirmed CD63 as a positive exosome marker. Epithelium-derived exosomes, when co-cultured with dental mesenchyme cells, robustly upregulated dentin sialophosphoprotein (DSPP) (SEQ ID NO: 33) expression (e.g., 20-fold increase), which is a pivotal dentinogenesis marker. Mass spectrometry identified multiple dozens of proteins in either epithelium- or mesenchyme-derived exosomes. MicroRNA arrays identified dozens of miRNAs in either epithelium- or mesenchyme-derived exosomes. For example, characterization of exosome proteins yielded Cofilin-1 and Periostin, which are involved in actin-modulation and odontogenesis. Quantitatively, exosome miRNAs differed significantly from miRNAs expressed by their parent cells. For example, compared to the microRNA profile in parental cells, miR-23a and miR-150, which are micro-RNAs that regulate tooth development and angiogenesis respectively, are enriched in exosomes. These and other findings described herein show that exosomes (e.g., from epithelium or mesenchyme) can mediate crosstalk between two cell types, as illustrated here in tooth development.

As described herein, exosomes can be used to promote dentinogenesis. Dentinogenesis is understood to be the formation of dentin. Dentinogenesis is understood to occur via odontoblasts, a type of biological cell on the outside of dental pulps. Dentin formation can result in different types of dentin including mantle dentin, primary dentin, secondary dentin, and tertiary dentin. In various embodiments, an exosome can be used to promote formation of one or more of mantle dentin, primary dentin, secondary dentin, or tertiary dentin.

As described herein, exosomes can be used to promote amelogenesis. Amelogenin is a protein product of ameloblasts in enamel formation and critical to the structure and mineralization of enamel in development. Amelogenin isoforms comprise −90% of the mineralized matrix that covers the crown of the tooth bud. As amelogenin is cleaved and degraded, mineral deposition in the form of crystals takes place in a hierarchical pattern. During amelogenesis, an organic, protein-rich substance which comprises over 85% amelogenin is transformed into a completely mineralized architecture of hydroxyapatite of enamel.

As described herein, exosomes can be used to promote odontogenesis. An odontoblast is a biological cell of neural crest origin that is part of the outer surface of the dental pulp, and whose biological function is dentinogenesis, which is the creation of dentin, the substance under the tooth enamel.

US App Pub No. 2014/0093481, published on 3 Apr. 2014 is incorporated herein by reference in its entirety.

Exosomes

As described herein, exosomes from epithelium or mesenchyme can mediate crosstalk between two cell types, as illustrated herein with respect to tooth development. Dental tissue-derived exosomes can contain macromolecules that are selectively, rather than passively, taken from the intracellular environment (see e.g., Example 7). Using GW4869 to decrease exosome secretion, Rab27A siRNA, or Rab27B siRNA, it was shown that exosome deficiency can result in the delay of tooth development or reduce collagen (e.g., collagen IV) formation (see e.g., Example 10, Example 11). Furthermore, it is shown herein that exosomes can participate in the BMP and Wnt signaling pathway (see e.g., Example 12).

It is understood in the art that exosomes contain mRNA or microRNA, which can be delivered to another cell, and can be functional in this new location (see e.g., Valadi et al. 2007 Nature Cell biology 9, 654-659).

Accordingly, exosomes derived from dental tissue can be used to treat a subject for a mineralization injury, disease or disorder or for promoting dentinogenesis. For example, exosomes derived from dental tissue can be used to treat a subject for a mineralization injury, disease or disorder. As another example, Accordingly, exosomes derived from dental tissue can be used to promote dentinogenesis.

Isolation of an Exosome

Processes for identification, isolation, or characterization of an exosome are understood in the art (see e.g. Examples 2-5; Jensen 2010 RNA Exosome (Advances in Experimental Medicine and Biology Book 702), Springer, ISBN-10: 1441978402). It is understood in the art that exosomes contain mRNA or microRNA, which can be delivered to another cell, and can be functional in this new location (see e.g., Valadi et al. 2007 Nature Cell biology 9, 654-659). Except as otherwise noted herein, therefore, the process of the present disclosure can be carried out in accordance with such processes.

Surface protein CD63 can be used as a marker of exosomes (Hadi Valadi et al., 2007, Nature Cell Biology). DSPP (SEQ ID NO: 33) expression as a dentinogenesis marker. DSPP (SEQ ID NO: 33) expression as a dentinogenesis marker. Upregulation of Dspp of greater than 20-fold compared to control (see e.g., Example 6).

An exosome described herein can be derived from dental tissue. For example, an exosome can be derived from dental epithelium. As another example, an exosome can be derived from mesenchyme-derived exosomes.

Epithelium-Derived Exosomes.

An epithelium-derived exosome can be used in compositions or methods described herein. Dental epithelium exosomes can promote differentiation towards odontogenesis (see e.g., Example 9). Thus, dental epithelium can transmit odontogenic signal to mesenchyme via exosomes.

An epithelium-derived exosome can be for a variety of effects. Dental epithelial exosomes, or a miRNA, RNA, or polypeptide contained therein, can induce upregulation of Dspp (e.g., SEQ ID NO: 33), a key transcriptional factors for odontogenesis (see e.g., Example 9). Dental epithelial exosomes, or a miRNA, RNA, or polypeptide contained therein, can induce upregulation of osteocalcin (OCN) (e.g., SEQ ID NO: 47) (see e.g., Example 9). Dental epithelial exosomes, or a miRNA, RNA, or polypeptide contained therein, can induce increased expression of alkaline phosphatase (e.g., SEQ ID NO: 48) (see e.g., Example 9). Dental epithelial exosomes, or a miRNA, RNA, or polypeptide contained therein, can promote calcium deposition (see e.g., Example 9).

A epithelium-derived exosome can have a particle size of about 80 to about 120 nm. An epithelium-derived exosome can have an average particle size of about 95 nm to about 105 nm. An epithelium-derived exosome can have an average particle size of about 100 nm.

Mesenchyme-Derived Exosomes.

An mesenchyme-derived exosome can be used in compositions or methods described herein. Dental mesenchyme exosomes can promote differentiation towards amelogenesis (see e.g., Example 8). It is presently thought that dental mesenchyme can transmit amelogenic signal to epithelium via exosomes.

A mesenchyme-derived exosome can have a particle size of about 80 to about 120 nm. A mesenchyme-derived exosome can have an average particle size of about 110 nm to about 120 nm. A mesenchyme-derived exosome can have an average particle size of about 116 nm.

A exosome described above can be used in a composition or method described herein alone; in combination with one or more other exosomes, miRNA, RNA, or polypeptides; as isolated; modified to contain less than an endogenous complement of miRNA, RNA, or polypeptides; or modified to contain more than an endogenous complement of miRNA, RNA, or polypeptides, including additional endogenous molecules or additional exogenous molecules.

miRNA

As described herein, miRNA contained within a dental tissue-derived exosome can be used to promote dentinogenesis or treat a mineralization injury, disease or disorder. Exosome miRNAs can differ significantly from miRNAs expressed by their parent cells. As such, exosomes can be used to identify miRNA useful for approaches described herein. miRNA associated with the BMP or Wnt signaling pathway (e.g., WNT/beta-catenin signaling pathway) can be useful for approaches described herein.

A miRNA associated with exosomes from dental tissue can be used for a variety of effects associated with the exosome or for independent effects. A miRNA associated with exosomes from dental tissue can be used to treat a subject for a mineralization injury, disease or disorder or for promoting dentinogenesis. For example, a miRNA associated with exosomes from dental tissue can be used to treat a subject for a mineralization injury, disease or disorder. As another example, a miRNA associated with exosomes from dental tissue can be used to promote dentinogenesis.

Processes for identification and isolation of an miRNA are understood in the art (see e.g. Ochiya 2013 Circulating MicroRNAs: Methods and Protocols (Methods in Molecular Biology), Humana Press, ISBN-10: 1627034528). Exosome microRNA profiles can be determined according to conventional methods in the art (see e.g., Example 7). Except as otherwise noted herein, therefore, the process of the present disclosure can be carried out in accordance with such processes.

A miRNA useful in a composition or method described herein can be identified or isolated from an epithelium-derived exosomes (see e.g., TABLE 3).

A miRNA can include rno-miR-674-5p (SEQ ID NO: 1), or a miRNA having at least about 80% (e.g., at least about 85%, 90%, 95%, or 99%) sequence identity thereto and retaining an activity associated with the miRNA.

A miRNA can include rno-miR-199a-3p (SEQ ID NO: 2), or a miRNA having at least about 80% (e.g., at least about 85%, 90%, 95%, or 99%) sequence identity thereto and retaining an activity associated with the miRNA.

A miRNA can include rno-miR-23b-3p (SEQ ID NO: 3), or a miRNA having at least about 80% (e.g., at least about 85%, 90%, 95%, or 99%) sequence identity thereto and retaining an activity associated with the miRNA.

A miRNA can include rno-miR-200b-3p£° (SEQ ID NO: 4), or a miRNA having at least about 80% (e.g., at least about 85%, 90%, 95%, or 99%) sequence identity thereto and retaining an activity associated with the miRNA.

A miRNA can include rno-miR-25-3p (SEQ ID NO: 5), or a miRNA having at least about 80% (e.g., at least about 85%, 90%, 95%, or 99%) sequence identity thereto and retaining an activity associated with the miRNA.

A miRNA can include rno-miR-672-5p£° (SEQ ID NO: 6), or a miRNA having at least about 80% (e.g., at least about 85%, 90%, 95%, or 99%) sequence identity thereto and retaining an activity associated with the miRNA.

A miRNA can include rno-miR-103-3p£° (SEQ ID NO: 7), or a miRNA having at least about 80% (e.g., at least about 85%, 90%, 95%, or 99%) sequence identity thereto and retaining an activity associated with the miRNA.

A miRNA useful in a composition or method described herein can be identified or isolated from an mesenchyme-derived exosomes (see e.g., TABLE 4).

A miRNA can include rno-let-7c-5p£° (SEQ ID NO: 8), or a miRNA having at least about 80% (e.g., at least about 85%, 90%, 95%, or 99%) sequence identity thereto and retaining an activity associated with the miRNA.

A miRNA can include rno-let-7a-5p£° (SEQ ID NO: 9), or a miRNA having at least about 80% (e.g., at least about 85%, 90%, 95%, or 99%) sequence identity thereto and retaining an activity associated with the miRNA.

A miRNA can include rno-let-7d-5p£° (SEQ ID NO: 10), or a miRNA having at least about 80% (e.g., at least about 85%, 90%, 95%, or 99%) sequence identity thereto and retaining an activity associated with the miRNA.

A miRNA can include rno-miR-352£° (SEQ ID NO: 11), or a miRNA having at least about 80% (e.g., at least about 85%, 90%, 95%, or 99%) sequence identity thereto and retaining an activity associated with the miRNA.

A miRNA can include rno-miR-532-3p£° (SEQ ID NO: 12), or a miRNA having at least about 80% (e.g., at least about 85%, 90%, 95%, or 99%) sequence identity thereto and retaining an activity associated with the miRNA.

A miRNA can include rno-miR-181b-5p£° (SEQ ID NO: 13), or a miRNA having at least about 80% (e.g., at least about 85%, 90%, 95%, or 99%) sequence identity thereto and retaining an activity associated with the miRNA.

A miRNA can include rno-miR-23b-3p£° (SEQ ID NO: 14), or a miRNA having at least about 80% (e.g., at least about 85%, 90%, 95%, or 99%) sequence identity thereto and retaining an activity associated with the miRNA.

A miRNA can include rno-miR-93-5p£° (SEQ ID NO: 15), or a miRNA having at least about 80% (e.g., at least about 85%, 90%, 95%, or 99%) sequence identity thereto and retaining an activity associated with the miRNA.

A miRNA can include rno-miR-16-5p£° (SEQ ID NO: 16), or a miRNA having at least about 80% (e.g., at least about 85%, 90%, 95%, or 99%) sequence identity thereto and retaining an activity associated with the miRNA.

A miRNA can include rno-miR-103-3p£° (SEQ ID NO: 17), or a miRNA having at least about 80% (e.g., at least about 85%, 90%, 95%, or 99%) sequence identity thereto and retaining an activity associated with the miRNA.

A miRNA can include rno-miR-151-5p£° (SEQ ID NO: 18), or a miRNA having at least about 80% (e.g., at least about 85%, 90%, 95%, or 99%) sequence identity thereto and retaining an activity associated with the miRNA.

A miRNA can include rno-miR-99b-5p£° (SEQ ID NO: 19), or a miRNA having at least about 80% (e.g., at least about 85%, 90%, 95%, or 99%) sequence identity thereto and retaining an activity associated with the miRNA.

A miRNA useful in a composition or method described herein can be identified or isolated from association with the BMP or Wnt signaling pathway (e.g., WNT/beta-catenin signaling pathway) (see e.g., Example 12).

A miRNA can include rno-miR-135b-5p£° (SEQ ID NO: 20), or a miRNA having at least about 80% (e.g., at least about 85%, 90%, 95%, or 99%) sequence identity thereto and retaining an activity associated with the miRNA.

A miRNA can include rno-miR-200a-3p£° (SEQ ID NO: 21), or a miRNA having at least about 80% (e.g., at least about 85%, 90%, 95%, or 99%) sequence identity thereto and retaining an activity associated with the miRNA.

A miRNA can include rno-miR-200b-3p£° (SEQ ID NO: 22), or a miRNA having at least about 80% (e.g., at least about 85%, 90%, 95%, or 99%) sequence identity thereto and retaining an activity associated with the miRNA.

A miRNA can include rno-miR-200b-5p£° (SEQ ID NO: 23), or a miRNA having at least about 80% (e.g., at least about 85%, 90%, 95%, or 99%) sequence identity thereto and retaining an activity associated with the miRNA.

A miRNA can include rno-miR-200c-3p£° (SEQ ID NO: 24), or a miRNA having at least about 80% (e.g., at least about 85%, 90%, 95%, or 99%) sequence identity thereto and retaining an activity associated with the miRNA.

A miRNA can include rno-miR-21-3p£° (SEQ ID NO: 25), or a miRNA having at least about 80% (e.g., at least about 85%, 90%, 95%, or 99%) sequence identity thereto and retaining an activity associated with the miRNA.

A miRNA can include rno-miR-21-5p£° (SEQ ID NO: 26), or a miRNA having at least about 80% (e.g., at least about 85%, 90%, 95%, or 99%) sequence identity thereto and retaining an activity associated with the miRNA.

A miRNA can include rno-miR-15b-3p£° (SEQ ID NO: 27), or a miRNA having at least about 80% (e.g., at least about 85%, 90%, 95%, or 99%) sequence identity thereto and retaining an activity associated with the miRNA.

A miRNA can include rno-miR-15b-5p£° (SEQ ID NO: 28), or a miRNA having at least about 80% (e.g., at least about 85%, 90%, 95%, or 99%) sequence identity thereto and retaining an activity associated with the miRNA.

A miRNA can include rno-miR-16-5p£° (SEQ ID NO: 29), or a miRNA having at least about 80% (e.g., at least about 85%, 90%, 95%, or 99%) sequence identity thereto and retaining an activity associated with the miRNA.

A miRNA can include rno-miR-122-5p£° (SEQ ID NO:30), or a miRNA having at least about 80% (e.g., at least about 85%, 90%, 95%, or 99%) sequence identity thereto and retaining an activity associated with the miRNA.

A miRNA can include rno-miR-203a-3p£° (SEQ ID NO: 31), or a miRNA having at least about 80% (e.g., at least about 85%, 90%, 95%, or 99%) sequence identity thereto and retaining an activity associated with the miRNA.

A miRNA can include rno-miR-375-3p£° (SEQ ID NO: 32), or a miRNA having at least about 80% (e.g., at least about 85%, 90%, 95%, or 99%) sequence identity thereto and retaining an activity associated with the miRNA.

A miRNA described herein can be used in a composition or method described herein alone, in combination with one or more other miRNA, RNA, or polypeptides, or in an exosome.

A miRNA described herein can be included in an expression vector, expression construct, plasmid, or recombinant nucleic acid construct. A vector, construct, or plasmid can include a transcribable nucleic acid molecule capable of being transcribed into a miRNA described herein. A transcribable nucleic acid molecule encoding a miRNA described herein can be operably linked to a promoter (e.g., an inducible promoter) functional in vitro or in vivo according to the species of the subject. A transcribable nucleic acid molecule encoding a miRNA described herein can be operably linked to a regulatory sequence.

A vector, construct, or plasmid encoding a miRNA described herein can be used to transform a host cell (e.g., in vitro transformation, ex vivo transformation, or in vivo transformation). A host cell transformed with a vector, construct, or plasmid encoding a miRNA described herein can be introduced (e.g., implanted) into a subject according to conventional techniques.

Polypeptide

As described herein, a polypeptide contained within a dental tissue-derived exosome can be used to promote dentinogenesis or treat a mineralization injury, disease or disorder. Polypeptide complements can differ significantly from miRNAs expressed by their parent cells. As such, exosomes can be used to identify polypeptides useful for approaches described herein.

A polypeptide useful in a composition or method described herein can be identified or isolated from an epithelium-derived exosomes (see e.g., TABLE 1).

A polypeptide can include connective tissue growth factor (CTGF) (SEQ ID NO: 34), or a polypeptide having at least about 80% (e.g., at least about 85%, 90%, 95%, or 99%) sequence identity thereto or a functional fragment thereof and retaining an activity associated with the polypeptide.

A polypeptide can include peroxiredoxin-2 (SEQ ID NO: 35), or a polypeptide having at least about 80% (e.g., at least about 85%, 90%, 95%, or 99%) sequence identity thereto or a functional fragment thereof and retaining an activity associated with the polypeptide.

A polypeptide can include odontogenic ameloblast-associated protein precursor (SEQ ID NO: 36), or a polypeptide having at least about 80% (e.g., at least about 85%, 90%, 95%, or 99%) sequence identity thereto or a functional fragment thereof and retaining an activity associated with the polypeptide.

A polypeptide can include hemiferrin, transferrin-like protein (SEQ ID NO: 37), or a polypeptide having at least about 80% (e.g., at least about 85%, 90%, 95%, or 99%) sequence identity thereto or a functional fragment thereof and retaining an activity associated with the polypeptide.

A polypeptide can include CaBP1 (SEQ ID NO: 38), or a polypeptide having at least about 80% (e.g., at least about 85%, 90%, 95%, or 99%) sequence identity thereto or a functional fragment thereof and retaining an activity associated with the polypeptide.

A polypeptide can include follistatin-related protein 1 precursor (SEQ ID NO: 39), or a polypeptide having at least about 80% (e.g., at least about 85%, 90%, 95%, or 99%) sequence identity thereto or a functional fragment thereof and retaining an activity associated with the polypeptide.

A polypeptide can include cofilin-1 (SEQ ID NO: 40), or a polypeptide having at least about 80% (e.g., at least about 85%, 90%, 95%, or 99%) sequence identity thereto or a functional fragment thereof and retaining an activity associated with the polypeptide.

A polypeptide useful in a composition or method described herein can be identified or isolated from an mesenchyme-derived exosomes (see e.g., TABLE 2).

A polypeptide can include annexin II (SEQ ID NO: 41), or a polypeptide having at least about 80% (e.g., at least about 85%, 90%, 95%, or 99%) sequence identity thereto or a functional fragment thereof and retaining an activity associated with the polypeptide.

A polypeptide can include lactadherin isoform b precursor (SEQ ID NO: 42), or a polypeptide having at least about 80% (e.g., at least about 85%, 90%, 95%, or 99%) sequence identity thereto or a functional fragment thereof and retaining an activity associated with the polypeptide.

A polypeptide can include pigment epithelium-derived factor precursor (SEQ ID NO: 43), or a polypeptide having at least about 80% (e.g., at least about 85%, 90%, 95%, or 99%) sequence identity thereto or a functional fragment thereof and retaining an activity associated with the polypeptide.

A polypeptide can include tenascin-N precursor (SEQ ID NO: 44), or a polypeptide having at least about 80% (e.g., at least about 85%, 90%, 95%, or 99%) sequence identity thereto or a functional fragment thereof and retaining an activity associated with the polypeptide.

A polypeptide can include keratin, type II cytoskeletal 5 (SEQ ID NO: 45), or a polypeptide having at least about 80% (e.g., at least about 85%, 90%, 95%, or 99%) sequence identity thereto or a functional fragment thereof and retaining an activity associated with the polypeptide.

A polypeptide can include periostin isoform 1 precursor (SEQ ID NO: 46), or a polypeptide having at least about 80% (e.g., at least about 85%, 90%, 95%, or 99%) sequence identity thereto or a functional fragment thereof and retaining an activity associated with the polypeptide.

A polypeptide described herein can be used in a composition or method described herein alone, in combination with one or more other miRNA, RNA, or polypeptides, or in an exosome.

A polypeptide described herein can be encoded by an expression vector, expression construct, plasmid, or recombinant nucleic acid construct. A vector, construct, or plasmid can include a transcribable nucleic acid molecule capable of being transcribed into a polypeptide described herein. A transcribable nucleic acid molecule encoding a polypeptide described herein can be operably linked to a promoter (e.g., an inducible promoter) functional in vitro or in vivo according to the species of the subject. A transcribable nucleic acid molecule encoding a polypeptide described herein can be operably linked to a regulatory sequence.

A vector, construct, or plasmid encoding a polypeptide described herein can be used to transform a host cell (e.g., in vitro transformation, ex vivo transformation, or in vivo transformation). A host cell transformed with a vector, construct, or plasmid encoding a polypeptide described herein can be introduced (e.g., implanted) into a subject according to conventional techniques.

Molecular Engineering

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Green and Sambrook 2012 Molecular Cloning: A Laboratory Manual, 4th ed., Cold Spring Harbor Laboratory Press, ISBN-10: 1605500569; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Green and Sambrook 2012 Molecular Cloning: A Laboratory Manual, 4th ed., Cold Spring Harbor Laboratory Press, ISBN-10: 1605500569; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A constructs of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of variant nucleotides or polypeptides having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l ). Furthermore, the Tₘ of a DNA:DNA hybrid is decreased by 1-1.5° C. for every 1% decrease in nucleotide identity (see e.g., Sambrook and Russell, 2006).

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Described herein are exosomes secreted by epithelium cells, mesenchyme cells, or mesoderm cells, or specific polypeptides or RNA contained therein or identified or isolated therefrom, that can act as diagnostic and therapeutic agents in a broad range of diseases and trauma.

Provided in the present disclosure is a process of treating a mineralization injury, disease or disorder in a subject in need administration of a therapeutically effective amount of composition or construct described herein, so as to increase mineralization in a target structure, tissue, or organ (e.g., promote dentinogenesis).

Processes for use of exosomes, RNA, or miRNA therapeutically are understood in the art (see e.g., Wood 2014 Exosome Biology and Therapeutics, Wiley-Blackwell, ISBN-10: 1118335805, providing a retrospective review; Jensen 2010 RNA Exosome (Advances in Experimental Medicine and Biology Book 702), Springer, ISBN-10: 1441978402; Sarkar 2014 MicroRNA Targeted Cancer Therapy, Springer, ASIN: B00JVIIWDQ; Lawrie 2013 MicroRNAs in Medicine, Wiley-Blackwell, ASIN: B00H6HIQVU; Guo and Hague 2013 RNA Nanotechnology and Therapeutics, CRC Press, ASIN: B00DJIVU0O). Except as otherwise noted herein, therefore, the process of the present disclosure can be carried out in accordance with such processes.

A determination of the need for treatment will typically be assessed by a history and physical exam consistent with the structure, tissue or organ defect at issue. Subjects with an identified need of therapy include those with a diagnosed mineralized structure, tissue or organ defect. As an example, a defect may include bone fracture, tooth extraction sockets, periodontal defects, non-unions, dental and orthopedic implant integration, and bony augmentation in reconstructive and plastic procedures. The subject is preferably an animal, including, but not limited to, mammals, reptiles, and avians, more preferably horses, cows, dogs, cats, sheep, pigs, and chickens, and most preferably human.

As an example, a subject in need may have a mineralized deficiency of at least 5%, 10%, 25%, 50%, 75%, 90% or more of a particular structure, tissue, or organ. As another example, a subject in need may have damage to a mineralized structure of a tissue or organ, and the method provides an increase in biological function by at least 5%, 10%, 25%, 50%, 75%, 90%, 100%, or 200%, or even by as much as 300%, 400%, or 500%. As yet another example, the subject in need may have a mineralization-related disease, disorder, or condition, and the method provides a mineralized engineered tissue or organ construct sufficient to ameliorate or stabilize the disease, disorder, or condition. In a further example, the subject in need may have an increased risk of developing a mineralization-related disease, disorder, or condition that is delayed or prevented by the method.

A composition described herein can be used to promote dentinogenesis. For example, compositions and methods described herein can increase dentinogenesis (e.g., formation of mantle dentin, primary dentin, secondary dentin, or tertiary dentin) by at least about 10%, 20%, 30%, 40%, 50%, 60%, 70%, 80%, 90%, 100%, 150%, 200%, 300%, 400%, 500%, 1000%, or more, as compared to a control.

A composition described herein can be used to promote amelogenesis. For example, compositions and methods described herein can increase amelogenesis by at least about 10%, 20%, 30%, 40%, 50%, 60%, 70%, 80%, 90%, 100%, 150%, 200%, 300%, 400%, 500%, 1000%, or more, as compared to a control.

A composition described herein can be used to promote odontogenesis. For example, compositions and methods described herein can increase odontogenesis by at least about 10%, 20%, 30%, 40%, 50%, 60%, 70%, 80%, 90%, 100%, 150%, 200%, 300%, 400%, 500%, 1000%, or more, as compared to a control.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to exosomes, polypeptides, or miRNA described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### Exosome Markers

The following examples describe the roles of exosomes in the crosstalk between epithelium and mesenchyme cells during tooth development, as a model of cell-cell communication. The Examples suggest that exosomes from epithelium and mesenchyme mediate crosstalk between two cell types, as illustrated below in tooth development.

### Example 2

### Exosome Isolation

The following example describes the protocol for exosome isolation from tooth epithelial and mesenchymal cells.

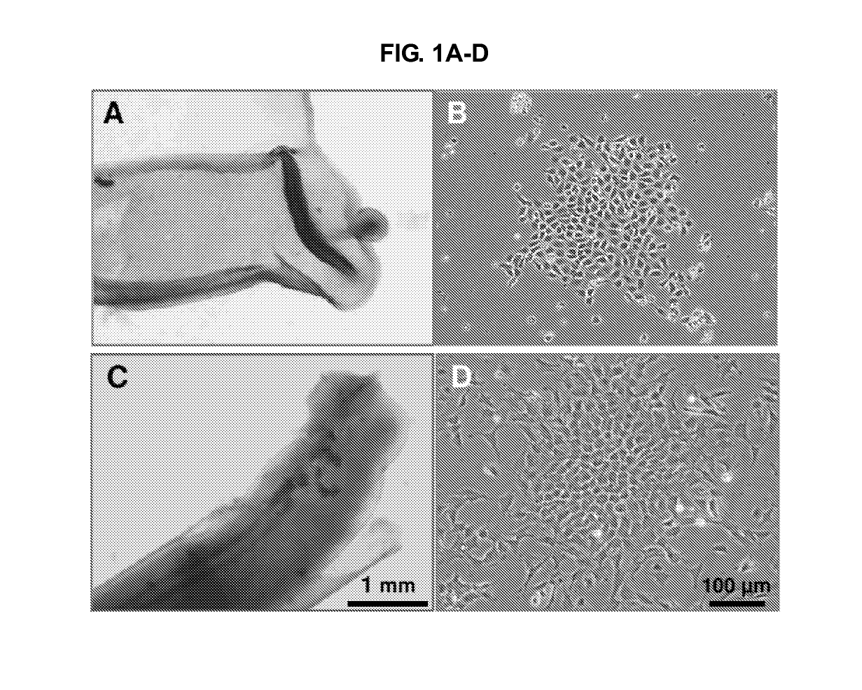

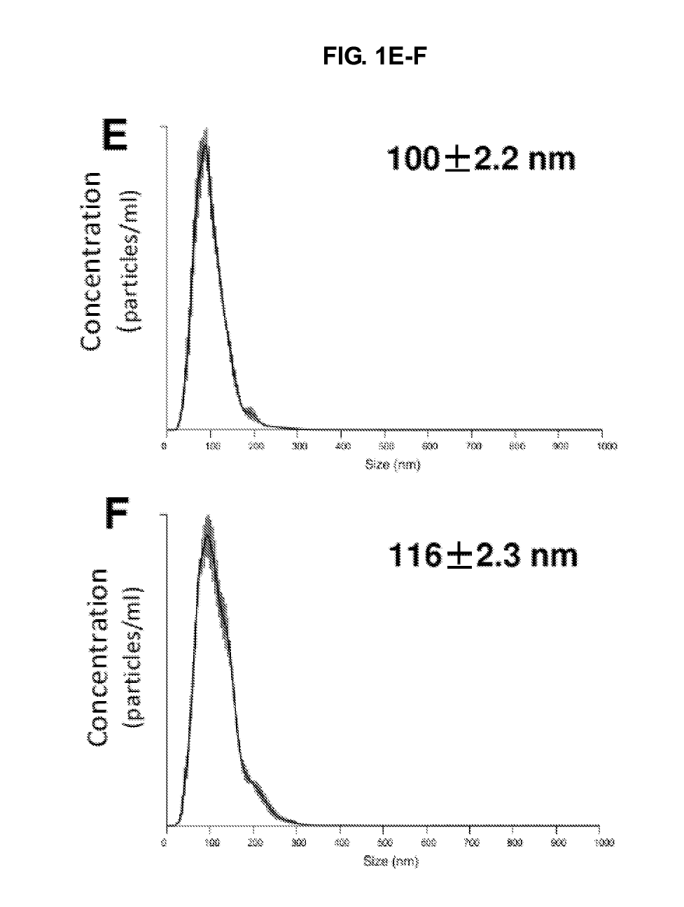

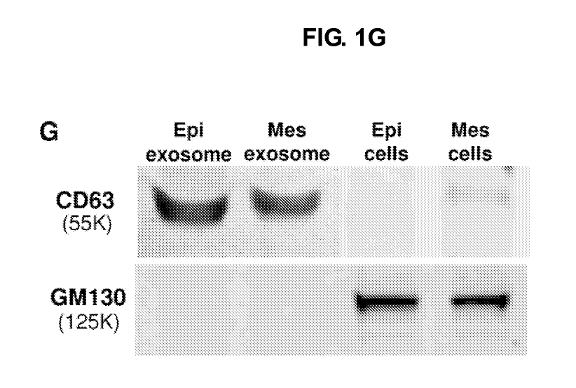

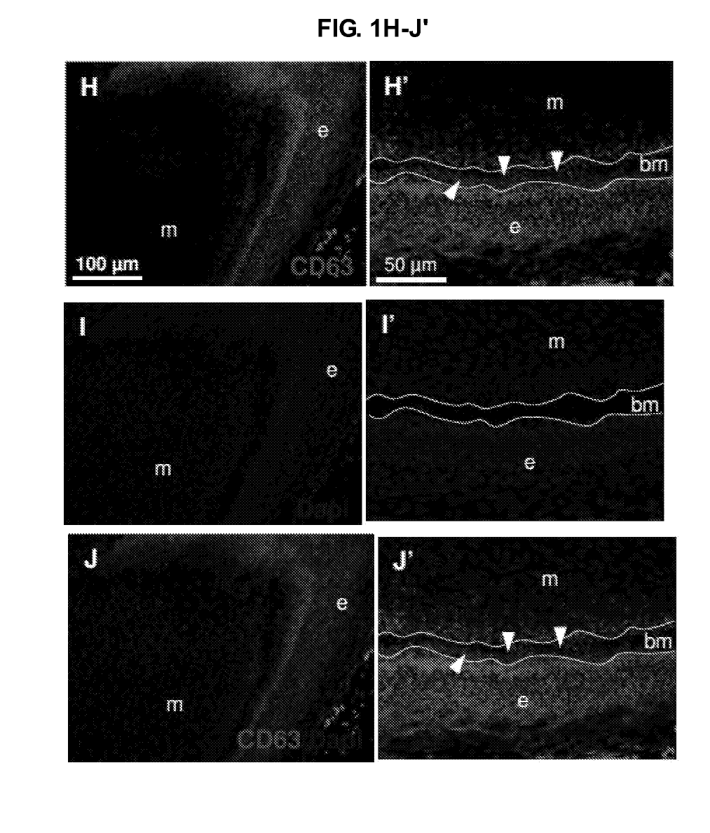

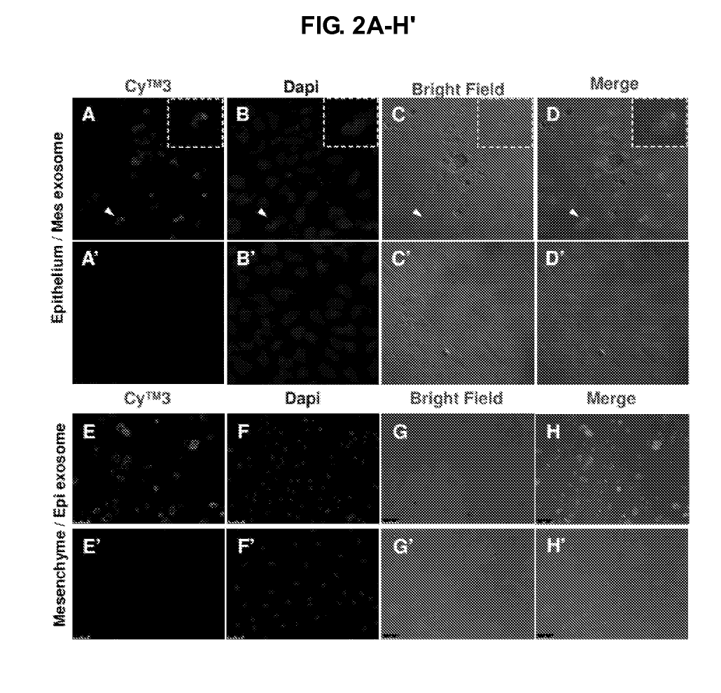

Dental epithelium (FIG. 1A) was dissected from dental mesenchyme (FIG. 1C) under dissection microscope. Stem/progenitor cells from dental epithelium (FIG. 1B) and mesenchyme (FIG. 1D) were propagated in exosome free media (KO medium for mesenchymal cells and LHC-8 for epithelial cells) at Day 3 for 1 week (see FIG. 1D-E).

Vesicles from culture medium were harvested by ultracentrifuge for further analysis, for example, for size analysis, Western Blot, RT-PCR, and Protein Assays. The supernatant was harvested and exosomes secreted by both cell types were isolated using ExoQuick exosome precipitation reagent (SBI).

### Example 3

### Size Analysis of Exosomes

The following example describes the identification and characterization of exosomes by size analysis using nanoparticle tracking analysis (NTA).

Epithelium- or mesenchyme-derived vesicles were separately isolated and verified to be in the range of 80-120 nm using nanoparticle tracking analysis (NTA) (see e.g., FIG. 1E-F). Specifically, the average diameter of particles purified from dental epithelial cell cultures was 100 nm and the average diameter of particles purified from mesenchymal cell cultures supernatant was 116 nm. The average particle sizes for both epithelial and mesenchymal cell cultures fell within the accepted range of exosome size.

### Example 4

### Western Blot Analysis of Exosomes

The following example describes the identification and characterization of exosomes using Western Blot.

The surface protein, CD63, is a commonly used marker of exosomes (Hadi Valadi et al., 2007, Nature Cell Biology). Western blotting showed epithelium (e) and adjacent mesenchyme (m) expressed CD63, especially in the cervical loop (FIG. 1H, I, J).

### Example 5

### Silver Staining and Mass Spectrometry of Proteins

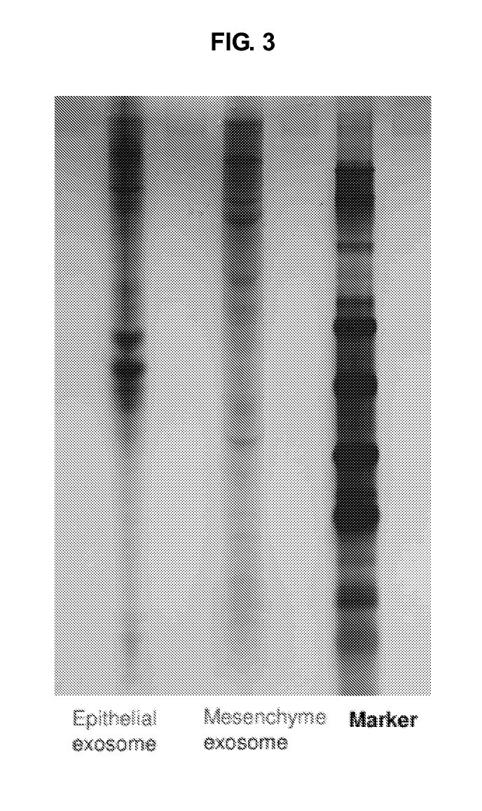

The following example describes the analysis of proteins using silver staining and mass spectrometry (see e.g., FIG. 3). Proteins extracted from epithelium and mesenchyme exosomes were loaded onto a 4-12% SDS-PAGE gel, followed by silver staining. Bands were cut from the gel according to molecular weight, and analyzed by mass spectrometry. Mass spectrometry identified multiple dozens of proteins in either epithelium- or mesenchyme-derived exosomes (see e.g., TABLE 1 and TABLE 2).

Epithelial exosome proteins were identified and characterized using silver staining and mass spectrometry (see e.g., TABLE 1). Cofilin-1 is overexpressed in dental epithelial exosomes and is an intracellular actin-modulating protein. Cofilin may play a role in the induction of cellular polarization in dental mesenchymal cells.

TABLE 1
Epithelial Exosome proteins

| | | |
|---|---|---|
| connective | 13386 | CTGF has important roles in many biological |
| tissue growth | | processes, including cell adhesion, migration, |
| factor (CTGF) | | proliferation, angiogenesis, skeletal |
| | | development, and tissue wound repair. |
| peroxiredoxin- | 21941 | The encoded protein may play an |
| 2 | | antioxidant protective role in cells, and may |
| | | contribute to the antiviral activity of CD8(+) |
| | | T-cells. |
| odontogenic | 30424 | Tooth-associated epithelia protein that |
| ameloblast- | | probably plays a role in odontogenesis, the |
| associated | | complex process that results in the initiation |
| protein | | and generation of the tooth. May be |
| precursor | | incorporated in the enamel matrix at the end |
| | | of mineralization process. |
| hemiferrin, | 24874 | the protein could fold somewhat like the C- |
| transferrin-like | | terminal lobe of transferrins |
| protein | | |
| CaBP1 | 47590 | Calcium binding proteins are an important |
| | | component of calcium mediated cellular |
| | | signal transduction. |
| follistatin- | 35740 | modulate the action of some growth factors |
| related protein | | on cell proliferation and differentiation. |
| 1 precursor | | |

Mesenchymal exosome proteins were identified using silver staining and mass spectrometry (see e.g., Table 2). Periostin is overexpressed in dental mesenchymal exosomes and has recently been implicated in regulating tooth formation and mineralization.

TABLE 2
Mesenchymal Exosome Proteins

| | | |
|---|---|---|
| annexin II | 39236 | Annexin2 is involved in diverse cellular processes such as cell |
| | | motility (especially epithelial cells), linkage of membrane-associated |
| | | protein complexes to the actin cytoskeleton, endocytosis, fibrinolysis, |
| | | ion channel formation, and cell matrix interactions. It is a Ca- |
| | | dependent phospholipid-binding protein whose function is to help |
| | | organize exocytosis of intracellular proteins to the extracellular |
| | | domain. |
| lactadherin | 48522 | Plays an important role in the maintenance of intestinal epithelial |
| isoform 2 | | homeostasis and the promotion of mucosal healing. Promotes |
| precursor | | VEGF-dependent neovascularization. Contributes to phagocytic |
| | | removal of apoptotic cells in many tissues. |
| pigment | 46493 | Neurotrophic protein; induces extensive neuronal differentiation in |
| epithelium-derived | | retinoblastoma cells. Potent inhibitor of angiogenesis. |
| factor precursor | | |
| tenascin-N | 174967 | Involved in neurite outgrowth and cell migration in hippocampal |
| precursor | | explants |
| keratin, type II | 62060 | This type II cytokeratin is specifically expressed in the basal layer of |
| cytoskeletal 5 | | the epidermis with family member KRT14. |
| Periostin | 90879 | osteoblast specific factor (predicted) |

### Example 6

### Gene Expression in Exosome Co-Culture

The following example describes gene expression in exosome co-cultures.

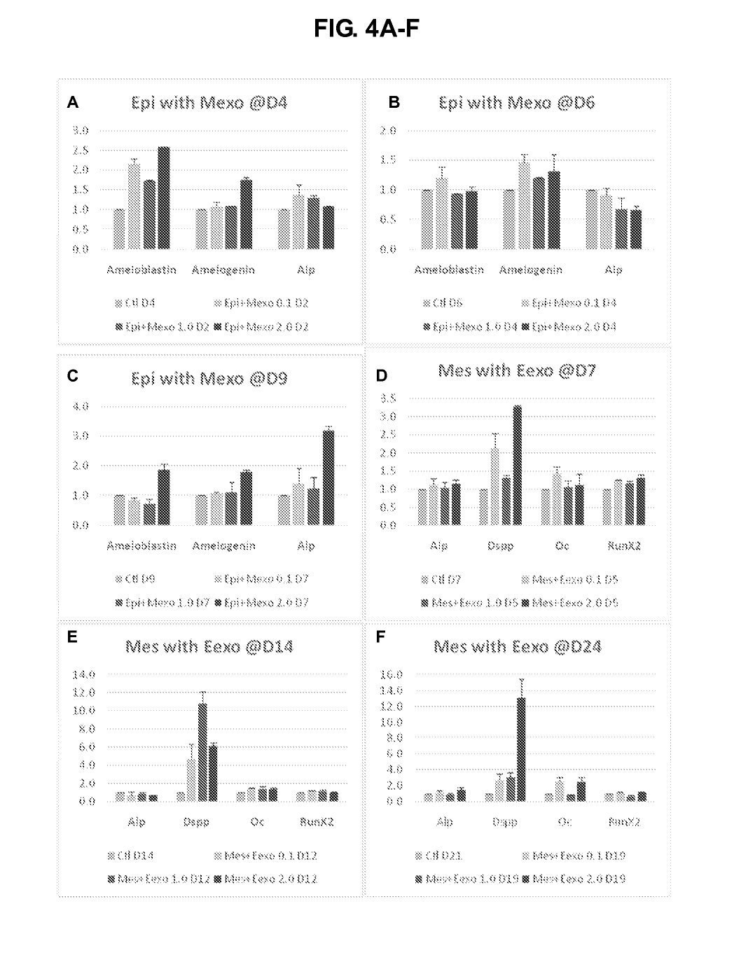

Epithelium-derived exosomes, when co-cultured with dental mesenchyme cells, robustly upregulated DSPP (SEQ ID NO: 33) expression, suggesting DSPP (SEQ ID NO: 33) could be a pivotal dentinogenesis marker (see e.g., FIG. 4A-F).

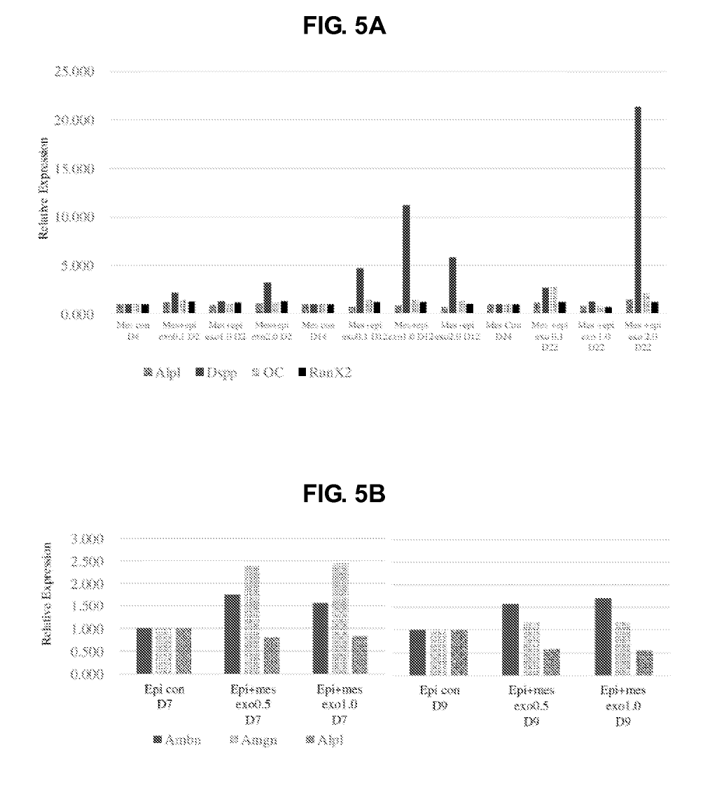

The following describes RT-PCR data from the differentiation analysis experiments. Relative expressions of alkaline phosphatase (Alpl), dentin sialophosphoprotein (Dspp), osteocalcin (OC), and Runt-related transcription factor 2 (RunX2) are shown for mesenchymal cells originating from dental pulp (FIG. 5A) exposed to varying concentrations of dental epithelial exosomes and at multiple timepoints. There is striking upregulation of Dspp of greater than 20-fold compared to control. Relative expression of ameloblastin (Ambn), amelogenin (Amgn), and alkaline phosphatase (Alpl) are shown for dental epithelial cells (FIG. 5B) exposed to dental mesenchymal exosomes.

### Example 7

### miRNA Expression Profiles of Dental Mesenchymal Cell and Exosomes

The following example describes miRNA expression profiles of dental mesenchymal cell and exosomes.

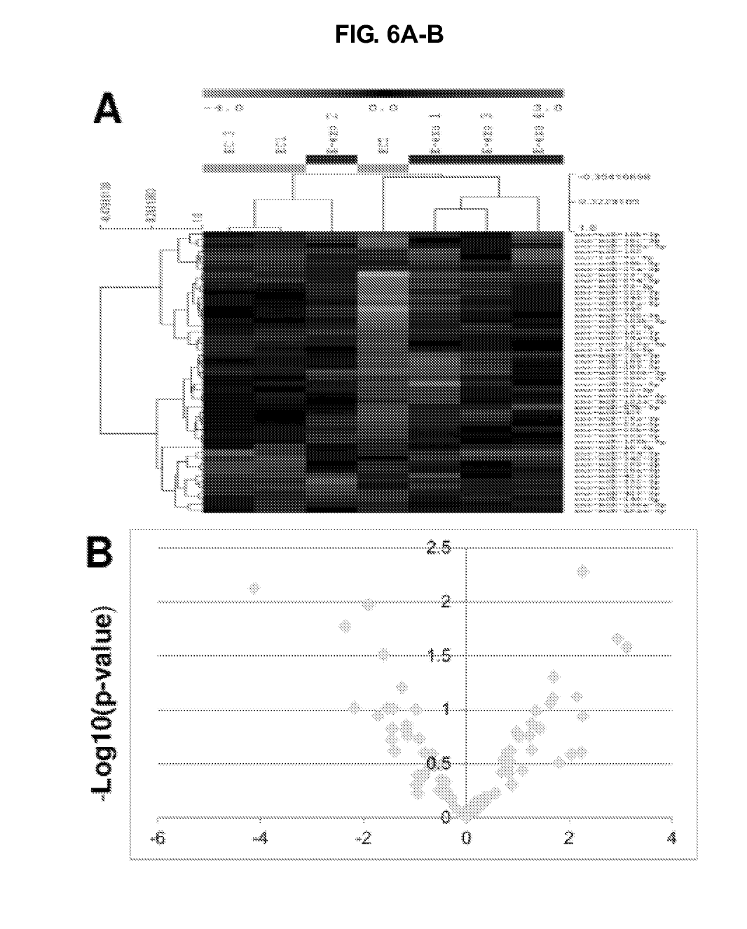

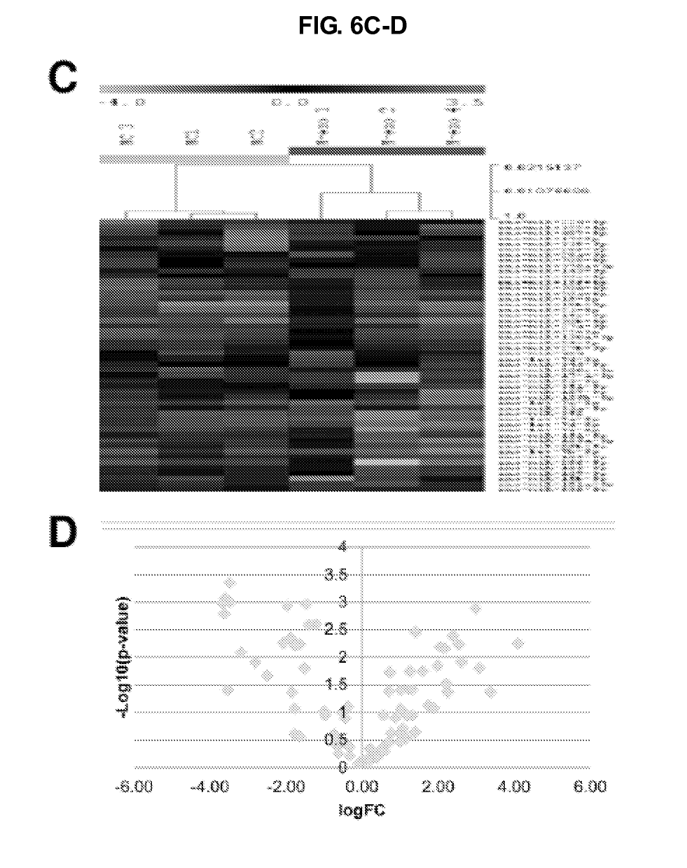

Total RNA was extracted using Trizol®. Samples of dental mesenchymal cells and exosomes were subjected to RT-PCR panel analysis for miRNA expression (RNA Universal RT microRNA PCR Services, Exiqon). The miRNA with greatest standard deviation in relative expression levels are depicted in the heat map diagram (see e.g., FIG. 6A, D). MicroRNA arrays identified dozens of miRNAs in either epithelium- or mesenchyme-derived exosomes. Quantitatively, exosome miRNAs differed significantly from miRNAs expressed by their parent cells (see e.g., FIG. 6B, D).

MicroRNA profiles of epithelial exosomes and their parental cells (dental epithelium stem/progenitor cells) were analyzed using microRNA array by miRCURY LNA™ and EXIQON (see e.g., FIG. 6A-B). TABLE 3 shows exemplary microRNAs in epithelium-derived exosomes.

MicroRNA profiles of epithelial exosomes and their parental cells (dental mesenchyme stem/progenitor cells) were analyzed using microRNA array by miRCURY LNA™ and EXIQON (see e.g., FIG. 6C-D). TABLE 4 shows arbitrarily selected microRNAs in mesenchyme-derived exosomes.

This expression data suggests that exosomes contain macromolecules that are selectively, rather than passively, taken from the intracellular environment.

TABLE 3
Differentially expressed microRNAs between
epithelial cells and their secreted exosomes.

| | Average | Average | | | | BH adj. |
|---|---|---|---|---|---|---|
| miR name | EC | exo | StDev | ddCp | p value | p-value |
| rno-miR-674-5p | −3.54 | −1.28 | 1.34 | 2.27 | 0.005 | 0.287 |
| rno-miR-199a-3p | 0.27 | −3.85 | 2.43 | −4.12 | 0.008 | 0.287 |
| rno-miR-23b-3p | 3.00 | 1.09 | 1.17 | −1.91 | 0.011 | 0.287 |
| rno-miR-200b-3p | 1.89 | −0.46 | 1.49 | −2.35 | 0.017 | 0.339 |
| rno-miR-25-3p | −4.47 | −1.53 | 1.90 | 2.94 | 0.022 | 0.353 |
| rno-miR-672-5p | −5.96 | −2.83 | 1.98 | 3.13 | 0.027 | 0.353 |
| rno-miR-103-3p | 1.42 | −0.18 | 1.07 | −1.60 | 0.031 | 0.353 |

TABLE 4
Differentially expressed microRNAs between
mesenchymal cells and their secreted exosomes.

| | | | | M-exo- | | BH p- |
|---|---|---|---|---|---|---|
| Name | MC | M-exo | SD | MC | t-test | value |
| rno-let-7c-5p | 4.03 | 0.56 | 1.93 | −3.47 | 0.0005 | 0.0158 |
| rno-let-7a-5p | 1.30 | −2.29 | 2.02 | −3.59 | 0.0009 | 0.0158 |
| rno-let-7d-5p | 1.56 | −1.91 | 1.95 | −3.47 | 0.0010 | 0.0158 |
| rno-miR-352 | −0.19 | −3.85 | 2.06 | −3.67 | 0.0011 | 0.0158 |
| rno-miR-532-3p | −0.57 | −2.03 | 0.83 | −1.47 | 0.0011 | 0.0158 |
| rno-miR-181a-5p | 3.46 | 1.50 | 1.11 | −1.96 | 0.0012 | 0.0158 |
| rno-miR-20a-5p | −0.35 | 2.64 | 1.69 | 2.99 | 0.0013 | 0.0158 |
| rno-let-7b-5p | 5.83 | 2.22 | 2.05 | −3.61 | 0.0016 | 0.0172 |
| rno-miR-181b-5p | −0.34 | −1.54 | 0.69 | −1.20 | 0.0026 | 0.0221 |
| rno-miR-23b-3p | 3.42 | 2.03 | 0.80 | −1.39 | 0.0026 | 0.0221 |
| rno-miR-93-5p | 0.17 | 1.58 | 0.81 | 1.40 | 0.0036 | 0.0271 |
| rno-miR-16-5p | 0.00 | 2.38 | 1.38 | 2.39 | 0.0041 | 0.0271 |
| rno-miR-103-3p | 1.85 | −0.03 | 1.09 | −1.88 | 0.0044 | 0.0271 |
| rno-miR-151-5p | 0.01 | −1.86 | 1.09 | −1.87 | 0.0048 | 0.0271 |
| rno-miR-99b-5p | 2.42 | 0.34 | 1.21 | −2.08 | 0.0055 | 0.0271 |

### Example 8

### Dental Mesenchyme Exosomes Promote Differentiation Towards Amelogenesis

The following example shows that dental mesenchyme exosomes promote differentiation towards amelogenesis.

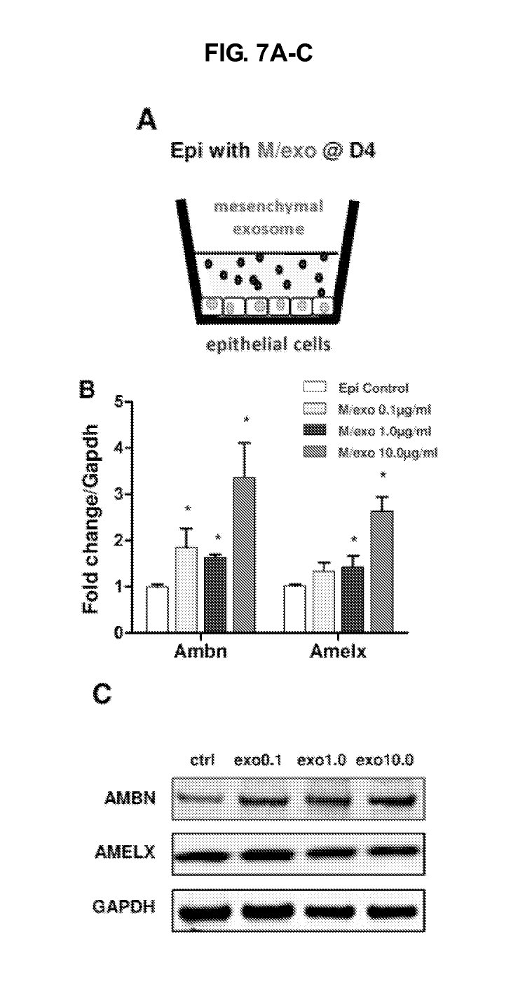

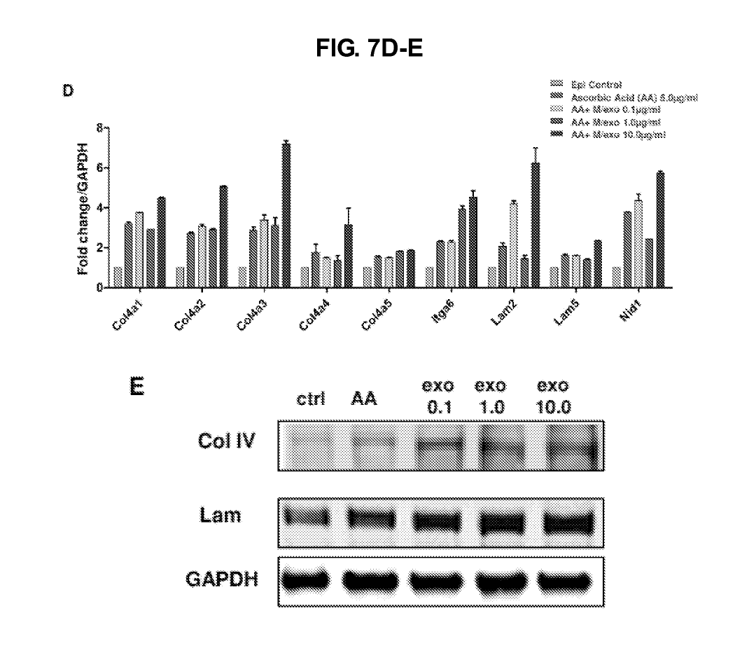

Dental epithelium stem/progenitor cells were incubated with exosomes secreted by dental mesenchyme stem/progenitor cells for 4 days (see e.g., FIG. 7A). Results showed that dental mesenchyme exosomes induced upregulation of ameloblastin (AMBN) and amelogenin (AMELX) at gene level (see e.g., FIG. 7B) and protein level (see e.g., FIG. 7C), key markers for amelogenesis.

Dental epithelium stem/progenitor cells were treated with dental mesenchyme exosomes with the presence of ascorbic acid (AA). Results showed upregulation of basement membrane components, such as Col4a, Itga, Iam and Nid, at gene level (see e.g., FIG. 7D) and protein level (see e.g., FIG. 7E).

These results suggest that dental mesenchyme transmits amelogenic signal to epithelium via exosomes.

### Example 9

### Dental Epithelium Exosomes Promote Differentiation Towards Odontogenesis

The following example shows that dental epithelium exosomes promote differentiation towards odontogenesis.

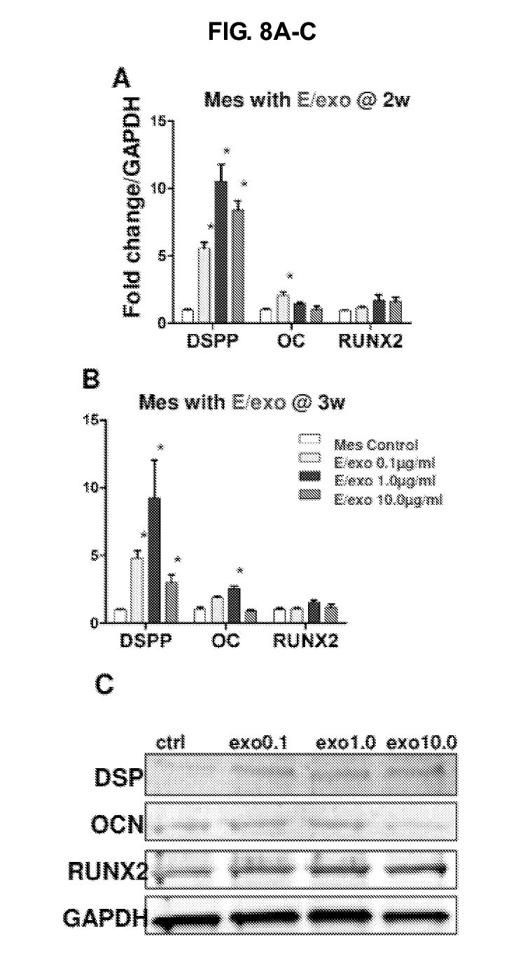

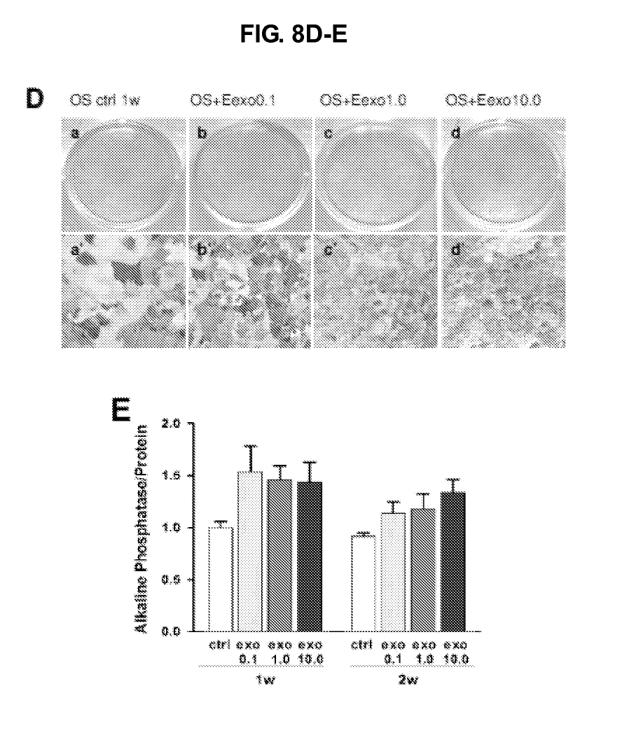

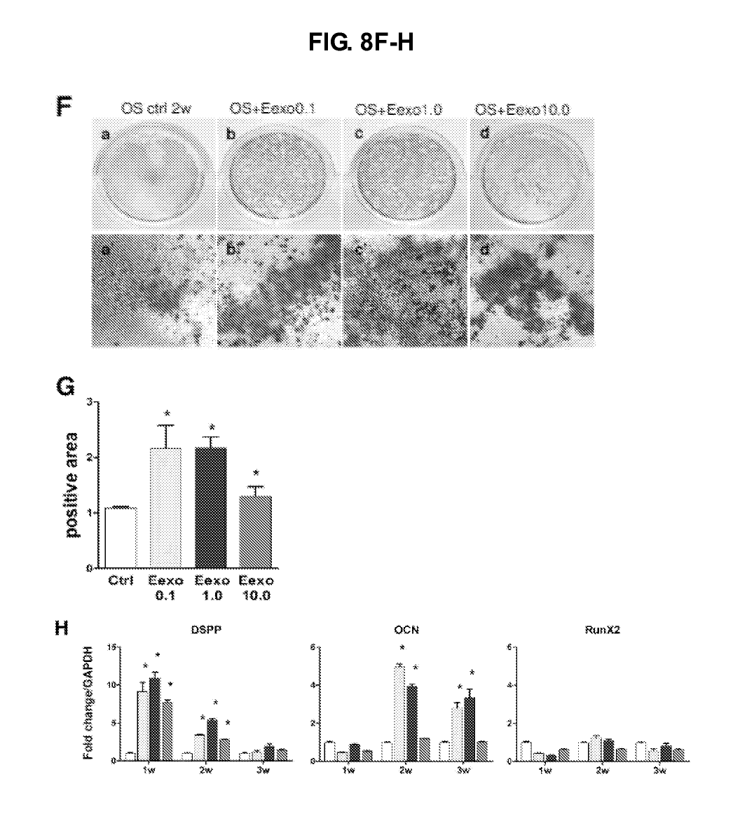

Expression levels of DSPP, OC, and RUNX2 were measured in dental mesenchyme stem/progenitor cells incubated with exosomes secreted by dental epithelium stem/progenitor cells for 14 (2 w) and 21 days (3 w). Results showed that dental epithelial exosomes induced robust upregulation of Dspp (See e.g., FIG. 8A-B). Western Blot showed an increase of DSP and OCN (see e.g., FIG. 8C).

Dental mesenchyme stem/progenitor cells were cultured in osteogenesis medium for one week. Results showed that dental epithelial exosomes induced increase expression of alkaline phosphatase expression (see e.g., FIG. 8D). Quantification of alkaline phosphatase expression was measured at 1 week and 2 weeks (see e.g., FIG. 8E).

Experiments with Alizarin Red staining showed epithelium exosomes (see e.g., FIG. 8F) promote calcium deposition (see e.g., FIG. 8G).

RT-PCR showed that dental epithelial exosomes induced robust upregulation of Dspp (see e.g., FIG. 8H), a key transcriptional factors for odontogenesis.

These data indicate that dental epithelium transmits odontogenic signal to mesenchyme via exosomes.

### Example 10

### Exosome Deficiency Resulted in the Delay of Tooth Development

The following examples shows that exosome deficiency resulted in the delay of tooth development.

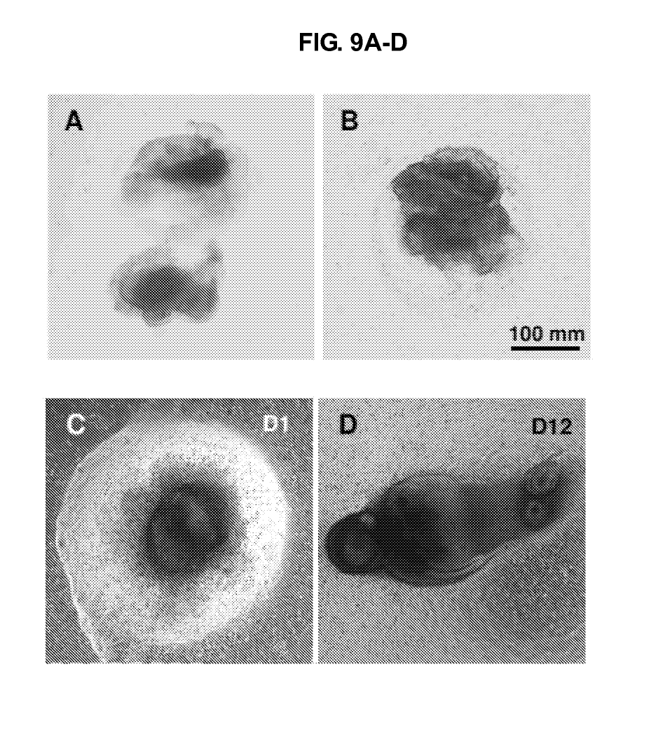

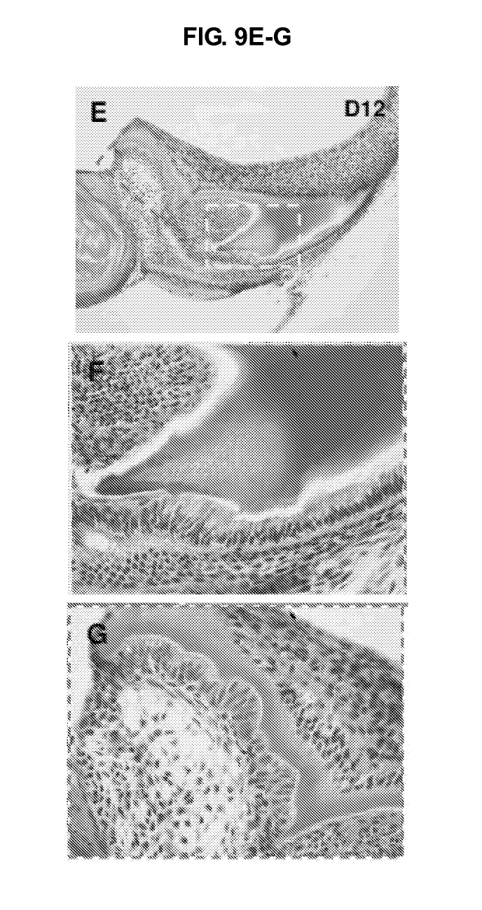

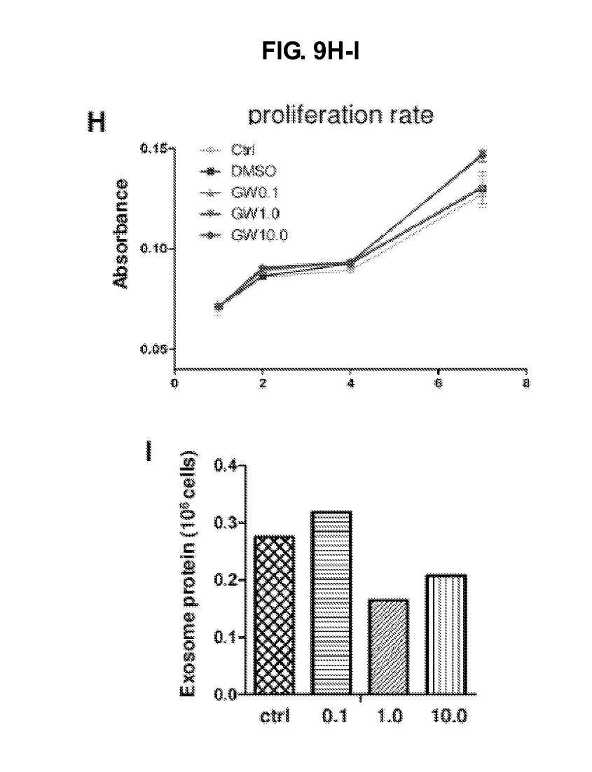

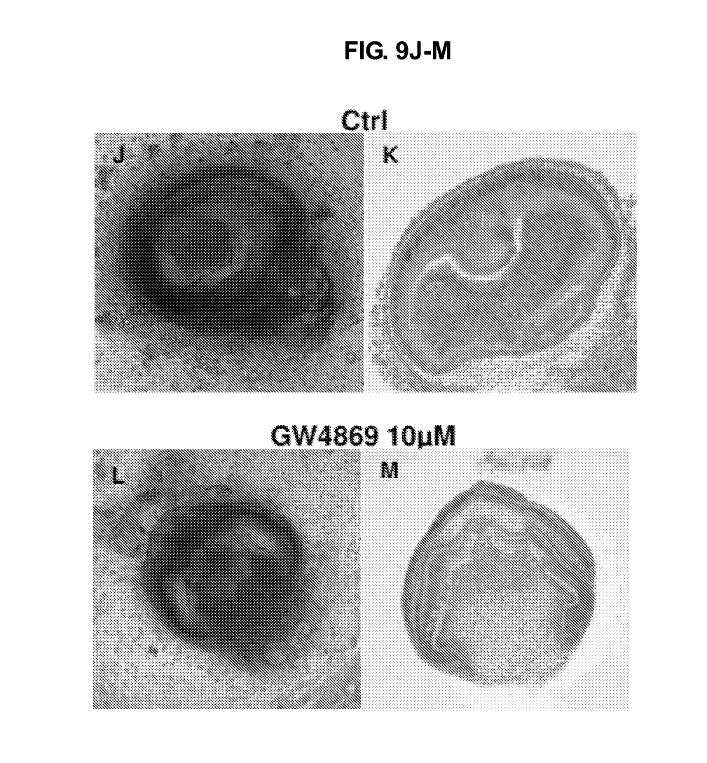

E16.5 epithelium and mesenchyme tissue (see e.g., FIG. 9A) were reconstituted (see e.g., FIG. 9B) under dissection microscope. The reconstituted organ were cultured for 12 days (see e.g., FIG. 9C-D).

Histology results showed robust dentin formation and cell polarization (see e.g., FIG. 9E-G). Exosome inhibitor GW4869 didn't affect the cell proliferation significantly (see e.g., FIG. 9H). GW4869 1.0 uM and 10.0 uM decreased the exosome secretion measured by protein concentration (see e.g., FIG. 9I). Organ culture showed dentin formation at day 10 in the control group (see e.g., FIG. 9J bright field, FIG. 9K, histological section followed by H&E staining). Dental epithelium and mesenchyme tissues were reconstituted in the presence of GW4869 10 uM (see e.g., FIG. 9L-M). No dentin formed (see e.g., FIG. 9M).

### Example 11

### Exosome Deficiency Resulted in the Delay of Tooth Development as Rab27A and Rab27B were Knocked Down

The following example shows that exosome deficiency resulted in the delay of tooth development as Rab27A and Rab27B were knocked down.

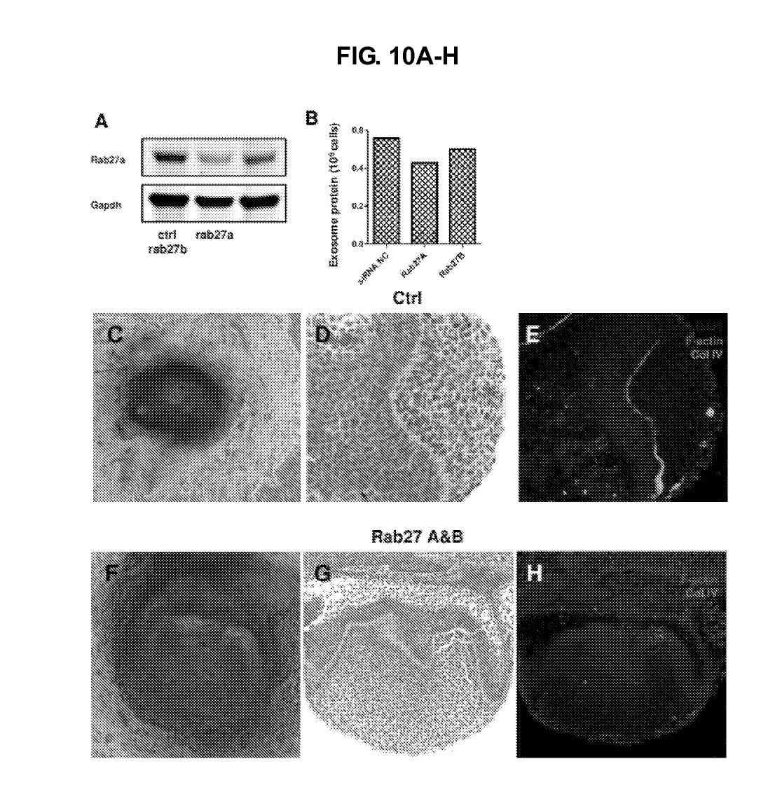

Dental mesenchyme cells were transfected with Rab27A and Rab27B siRNA (see e.g., FIG. 10A). The efficiency of knock down were measured by western blot and exosome secretion measured by protein concentration (see e.g., FIG. 10B). In FIG. 10C-E, Organ culture showed basement formation at day 4 in the control group (see e.g., FIG. 10C, bright field; FIG. 10D, histological section followed by H&E staining; FIG. 10E, immunofluorescence staining for type IV collagen). In FIG. 10E-H, Dental epithelium and mesenchyme tissues were reconstituted as Rab27A and Rab27B were knocked down (see e.g., FIG. 10E-H). Reduced collagen IV was detected (see e.g., FIG. 10H).

### Example 12

### Exosomes Participate in the BMP and Wnt Signaling Pathway

The following example shows exosomes participate in the BMP and Wnt signaling pathway.

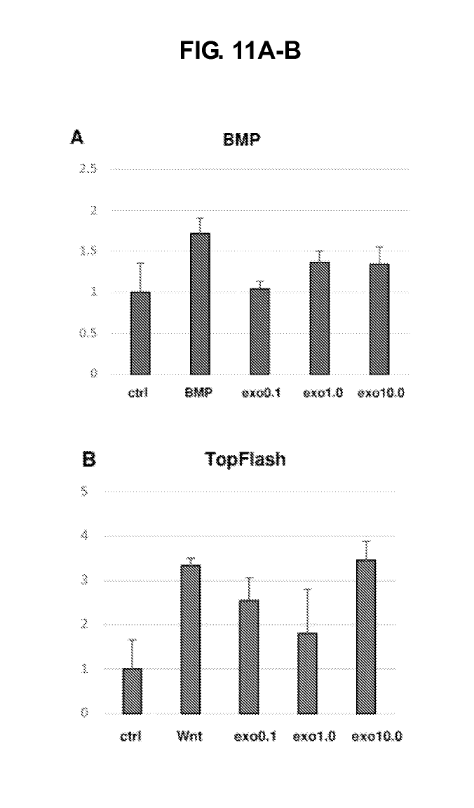

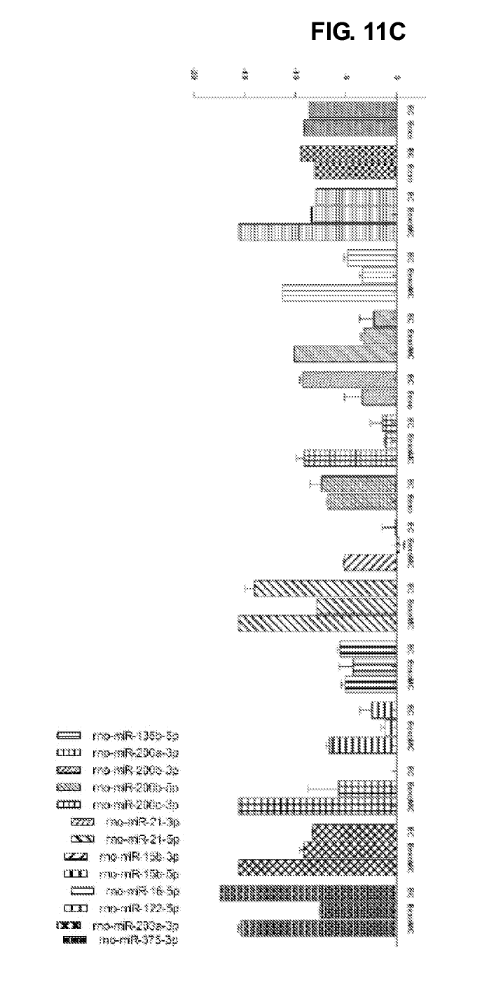

Assays were performed in dental mesenchyme stem/progenitor cells harboring either 12XSBE (BMP) or Topflash (Wnt) expression vector. Results showed that relative luciferase activities driven by epithelium stem/progenitor derived exosomes (see e.g., FIG. 11A-B). ΔCT value was measured selected miRNAs in epithelium cells (EC), epithelium-derived exosomes (Eexo) and mesenchyme cells (MC), which are related with WNT/beta-catenin signaling pathway (see e.g., FIG. 11C).

### LITERATURE CITED

Bobrie A, Colombo M, Raposo G, et al. Exosome secretion: Molecular mechanisms and roles in immune responses. Traffic 2011; 12: 1659-1668.
Lässer, C., Eldh, M., Lotvall, J. Isolation and Characterization of RNA-Containing Exosomes. J. Vis. Exp. (59), e3037 10.3791/3037.
Lin C W, Yen S T, Chang H T, et al. Loss of cofilin 1 disturbs actin dynamics, adhesion between enveloping and deep cell layers and cell movements during gastrulation in zebrafish. PLoS One. 2010 Dec. 22; 5(12):e15331.
Ludwig A K, Giebel B. Exosomes: small vesicles participating in intercellular communication. Int J Biochem Cell Biol. 2012 January; 44(1):11-5.
Ma D, Zhang R, Sun Y, et al. A novel role of periostin in postnatal tooth formation and mineralization. J Biol Chem. 2011 Feb. 11; 286(6):4302-9.
Pan B T, Teng K, Wu C, et al. Electron microscopic evidence for externalization of the transferring receptor in vesicular form in sheep reticulocytes, J. Cell Biol. 101 (1985) 942-948.
Thesleff I, Lehtonen E, Wartiovaara J, et al. Interference of tooth differentiation with interposed filters. Dev Biol 1977; 58: 197-203.
Valadi H, Ekstrom K, Bossios A, et al. Exosome-mediate transfer of mRNAs and microRNAs is a novel mechanism of genetic exchange between cells. Nat Cell Biol. 2007 June; 9(60):654-9.
Vlassov A V, Magdaleno S, Setterquist R, et al. Exosomes: Current knowledge of their composition, biological function, diagnostic and therapeutic potentials. Biochim Biophys Acta. 2012 July; 1820(7):940-8.

SEQUENCE LISTING

```
rno-miR-674-5p:
SEQ ID NO: 1
GCACUGAGAUGGGAGUGGUGUA
rno-miR-199a-3p:
SEQ ID NO: 2
ACAGUAGUCUGCACAUUGGUUA
rno-miR-23b-3p:
SEQ ID NO: 3
AUCACAUUGCCAGGGAUUACC
rno-miR-200b-3p£°
SEQ ID NO: 4
UAAUACUGCCUGGUAAUGAUGAC
rno-miR-25-3p:
SEQ ID NO: 5
CAUUGCACUUGUCUCGGUCUGA
rno-miR-672-5p£°
SEQ ID NO: 6
UGAGGUUGGUGUACUGUGUGUGA
rno-miR-103-3p£°
SEQ ID NO: 7
AGCAGCAUUGUACAGGGCUAUGA
rno-let-7c-5p£°
SEQ ID NO: 8
UGAGGUAGUAGGUUGUAUGGUU
rno-let-7a-5p£°
SEQ ID NO: 9
UGAGGUAGUAGGUUGUAUAGUU
rno-let-7d-5p£°
SEQ ID NO: 10
AGAGGUAGUAGGUUGCAUAGUU
rno-miR-352£°
SEQ ID NO: 11
AGAGUAGUAGGUUGCAUAGUA
rno-miR-532-3p£°
SEQ ID NO: 12
CCUCCCACACCCAAGGCUUGCA
rno-miR-181b-5p£°
SEQ ID NO: 13
AACAUUCAUUGCUGUCGGUGGGU
rno-miR-23b-3p£°
SEQ ID NO: 14
AUCACAUUGCCAGGGAUUACC
rno-miR-93-5p£°
SEQ ID NO: 15
CAAAGUGCUGUUCGUGCAGGUAG
rno-miR-16-5p£°
SEQ ID NO: 16
UAGCAGCACGUAAAUAUUGGCG
rno-miR-103-3p£°
SEQ ID NO: 17
AGCAGCAUUGUACAGGGCUAUGA
rno-miR-151-5p£°
SEQ ID NO: 18
UCGAGGAGCUCACAGUCUAGU
rno-miR-99b-5p£°
SEQ ID NO: 19
CACCCGUAGAACCGACCUUGCG
rno-miR-135b-5p£°
SEQ ID NO: 20
UAUGGCUUUUCAUUCCUAUGUGA
rno-miR-200a-3p£°
SEQ ID NO: 21
UAACACUGUCUGGUAACGAUGU
rno-miR-200b-3p£°
SEQ ID NO: 22
UAAUACUGCCUGGUAAUGAUGAC
rno-miR-200b-5p£°
SEQ ID NO: 23
CAUCUUACUGGGCAGCAUUGGA
rno-miR-200c-3p£°
SEQ ID NO: 24
UAAUACUGCCGGGUAAUGAUG
rno-miR-21-3p£°
SEQ ID NO: 25
CAACAGCAGUCGAUGGGCUGUC
rno-miR-21-5p£°
SEQ ID NO: 26
UAGCUUAUCAGACUGAUGUUGA
rno-miR-15b-3p£°
SEQ ID NO: 27
CGAAUCAUUAUUUGCUGCUCUA
rno-miR-15b-5p£°
SEQ ID NO: 28
UAGCAGCACAUCAUGGUUUACA
rno-miR-16-5p£°
SEQ ID NO: 29
UAGCAGCACGUAAAUAUUGGCG
rno-miR-122-5p£°
SEQ ID NO: 30
UGGAGUGUGACAAUGGUGUUUG
rno-miR-203a-3p£°
SEQ ID NO: 31
GUGAAAUGUUUAGGACCACUAG
rno-miR-375-3p£°
SEQ ID NO: 32
UUUGUUCGUUCGGCUCGCGUGA
dentin sialophosphoprotein precursor [Homo sapiens]
GenBank Accession No. AF163151.2
SEQ ID NO: 33
      1 mkiityfciw avawaipvpq skplerhvek smnlhllars nvsvqdelna sgtikesgvl
    61 vhegdrgrqe ntqdghkgeg ngskwaevgg ksfstystla neegniegwn gdtgkaetyg
  121 hdgihgkeen itangiqgqv siidnagatn rsntngntdk ntqngdvgda ghnedvavvq
  181 edgpqvagsn nstdnedeii enscrnegnt seitpqinsk rngtkeaevt pgtgedagld
  241 nsdgspsgng adededegsg ddedeeagng kdssnnskgq egqdhgkedd hdssigqnsd
  301 skeyydpegk edphnevdgd ktskseensa gipedngsqr iedtqklnhr eskrvenrit
  361 kesethavgk sqdkgieikg pssgnrnitk evgkgnegke dkgqhgmilg kgnvktqgev
  421 vniegpgqks epgnkvghsn tgsdsnsdgy dsydfddksm qgddpnssde sngnddanse
  481 sdnnsssrgd asynsdeskd ngngsdskga edddsdstsd tnnsdsngng nngnddndks
  541 dsgkgksdss dsdssdssns sdssdssdsd ssdsnsssds dssdsdssds sdsdssdssn
  601 ssdssdssds sdssdssdss dsksdsskse sdssdsdsks dssdsnssds sdnsdssdss
  661 nssnssdssd ssdssdssss sdsssssdss nssdssdssd ssnssessds sdssdsdssd
  721 ssdssnsnss dsdssnssds sdssdssdss nssdssdssd ssnssdssds sdssdssdss
  781 nssdsndssn ssdssdssns sdssnssdss dssdssdsds snssdssnss dssdssnssd
  841 ssdssdssds sdsdssnrsd ssnssdssds sdssnssdss dssdssdsne ssnssdssds
  901 snssdsdssd ssnssdssds snssdssess nssdnsnssd ssnssdssds sdssnssdss
  961 nsgdssnssd ssdsnssdss dssnssdssd ssdssdssds sdssnssdss dssdssdssn
1021 ssdssnssds sdssdssdss dssdssnssd ssdssdssds sdssgssdss dssdssdssd
1081 ssdssdssds sdssessdss dssdssdssd ssdssdssds sdssdssdss nssdssdssd
1141 ssdssdssds sdssdssdss dssdssdssd ssdssdssds sdsnessdss dssdssdssn
1201 ssdssdssds sdstsdsnde sdsqsksgng nnngsdsdsd segsdsnhst sdd
connective tissue growth factor (CTGF) [Homo sapiens]
ACCESSION CAG46534
SEQ ID NO: 34
      1 mtaasmgpvr vafvvllalc srpavgqncs gpcrypdepa prcpagvslv ldgcgccrvc
    61 akqlgelcte rdpcdphkgl fcdfgspanr kigvctakdg apcifggtvy rsgesfqssc
  121 kyqctcldga vgcmplcsmd vrlpspdcpf prrvklpgkc ceewvcdepk dqtvvgpala
  181 ayrledtfgp dptmirancl vqttewsacs ktcgmgistr vtndnascrl ekqsrlcmvr
  241 pceadleeni kkgkkcirtp kiskpikfel sgctsmktyr akfcgvctdg rcctphrttt
  301 lpvefkcpdg evmkknmmfi ktcachyncp gdndifesly yrkmygdma
peroxiredoxin-2 [Homo sapiens]
ACCESSION NP_005800
SEQ ID NO: 35
      1 masgnarigk papdfkatav vdgafkevkl sdykgkyvvl ffypldftfv cpteiiafsn
    61 raedfrklgc evlgvsvdsq fthlawintp rkegglgpin iplladvtrr lsedygvlkt
  121 degiayrglf iidgkgvlrq itvndlpvgr svdealrlvq afqytdehge vcpagwkpgs
  181 dtikpnvdds keyfskhn
odontogenic ameloblast-associated protein precursor [Homo sapiens]
ACCESSION NP_060325
SEQ ID NO: 36
      1 mkiiillgfl gatlsaplip qrlmsasnsn elllnlnngq llplqlqgpl nswippfsgi
    61 lqqqqqaqip glsqfslsal dqfagllpnq ipltgeasfa qgaqagqvdp lqlqtppqtq
  121 pgpshvmpyv fsfkmpqeqg qmfqyypvym vlpweqpqqt vprspqqtrq qqyeeqipfy
  181 aqfgyipqla epaisggqqq lafdpqlgta peiavmstge eipylqkeal nfrhdsagvf
  241 mpstspkpst tnvftsavdq titpelpeek dktdslrep
hemiferrin, transferrin-like protein [Rattus norvegicus]
ACCESSION NP_775443
SEQ ID NO: 37
      1 mlyskinnck fdeffsagca pgsprnsssl calcigsekg tgkecvpnsn eryygytgaf
    61 rclvekgdva fvkdqtviqn tdgnnneawa knmkkenfev lckdgtrkpv tdaenchlpe
  121 pnhavvsrkd katcvekiln kqqddfgksv tdctsnfclf qsnskdllfr ddtkclasia
  181 kktydsylgd dyvramtnlr qcstskllea ctfhkp
calcium-binding protein 1 isoform 2 (CaPB1) [Homo sapiens]
ACCESSION NP_004267
SEQ ID NO: 38
      1 mgncvkyplr nlsrkdrslr peeieelrea frefdkdkdg yincrdlgnc mrtmgympte
    61 melielsqqi nmnlgghvdf ddfvelmgpk llaetadmig vkelrdafre fdtngdgeis
  121 tselreamrk llghqvghrd ieeiirdvdl ngdgrvdfee fvrmmsr
follistatin-related protein 1 precursor [Homo sapiens]
ACCESSION NP_009016
SEQ ID NO: 39
      1 mwkrwlalal alvavawvra eeelrskski canvfcgagr ecavtekgep tclcieqckp
    61 hkrpvcgsng ktylnhcelh rdacltgski qvdydghcke kksyspsasp vvcyqsnrde
  121 lrrriiqwle aeiipdgwfs kgsnyseild kyfknfdngd srldsseflk fveqnetain
  181 ittypdqenn kllrglcvda lielsdenad wklsfqeflk clnpsfnppe kkcaledety
  241 adgaetevdc nrcvcacgnw vctamtcdgk nqkgaqtqte eemtryvqel qkhqetaekt
  301 krvstkei
cofilin-1 [Homo sapiens]
ACCESSION NM_005507.2
SEQ ID NO: 40
      1 masgvaysdg vikvfndmkv rksstpeevk krkkavlfcl sedkkniile egkeilvgdv
    61 gqtvddpyat fvkmlpdkdc ryalydatye tkeskkedlv fifwapesap lkskmiyass
  121 kdaikkkltg ikhelqancy eevkdrctla eklggsavis legkpl
annexin II [Homo sapiens]
ACCESSION P07355
SEQ ID NO: 41
      1 mstvheilck lslegdhstp psaygsvkay tnfdaerdal nietaiktkg vdevtivnil
    61 tnrsnaqrqd iafayqrrtk kelasalksa lsghletvil gllktpaqyd aselkasmkg
  121 lgtdedslie licsrtngel qeinrvykem yktdlekdii sdtsgdfrkl mvalakgrra
  181 edgsvidyel idqdardlyd agvkrkgtdv pkwisimter svphlqkvfd ryksyspydm
  241 lesirkevkg dlenaflnlv qciqnkplyf adrlydsmkg kgtrdkvlir imvsrsevdm
  301 lkirsefkrk ygkslyyyiq qdtkgdyqka llylcggdd
lactadherin isoform b precursor [Homo sapiens]
ACCESSION NP_001108086
SEQ ID NO: 42
      1 mprprllaal cgallcapsl lvaldicskn pchngglcee isqevrgdvf psytctclkg
    61 yagnhcetkc veplglengn iansqlaass vrvtflglqh wvpelarlnr agmvnawtps
  121 snddnpwiqv nllrrmwvtg vvtqgasrla sheylkafkv ayslnghefd fihdvnkkhk
  181 efvgnwnkna vhvnlfetpv eaqyvrlypt schtactlrf ellgcelngc anplglknns
  241 ipdkqitass syktwglhlf swnpsyarld kqgnfnawva gsygndqwlq ifpgnwdnhs
  301 hkknlfetpi laryvrilpv awhnrialrl ellgc
pigment epithelium-derived factor precursor [Homo sapiens]
ACCESSION NP_002606
SEQ ID NO: 43
      1 mqalvlllci gallghsscq npasppeegs pdpdstgalv eeedpffkvp vnklaaavsn
    61 fgydlyrvrs stspttnvll splsvatals alslgaeqrt esiihralyy dlisspdihg
  121 tykelldtvt apqknlksas rivfekklri kssfvaplek sygtrprvlt gnprldlqei
  181 nnwvqaqmkg klarstkeip deisilllgv ahfkgqwvtk fdsrktsled fyldeertvr
  241 vpmmsdpkav lrygldsdls ckiaqlpltg smsiifflpl kvtqnitlie esltsefihd
  301 idrelktvqa vltvpklkls yegevtkslq emklqslfds pdfskitgkp ikltqvehra
  361 gfewnedgag ttpspglqpa hltfpldyhl nqpfifvlrd tdtgallfig kildprgp
tenascin-N precursor [Homo sapiens]
ACCESSION NP_071376 XP_040527
SEQ ID NO: 44
      1 mslqemfrfp mglllgsvll vasapatlep pgcsnkeqqv tvshtykidv pksalvqvda
    61 dpqplsddga sllalgeare eqniifrhni rlqtpqkdce lagsvqdlla rvkkleeemv
  121 emkeqcsaqr ccqgvtdlsr hcsghgtfsl etcschceeg regpacerla cpgacsghgr
  181 cvdgrciche pyvgadcgyp acpencsghg ecvrgvcqch edfmsedcse krcpgdcsgh
  241 gfcdtgecyc eegftgldca qvvtpqglql lkntedsllv swepssqvdh yllsyyplgk
  301 elsgkqiqvp keqhsyeilg llpgtkyivt lrnvknevss spqhllattd lavlgtawvt
  361 detensldve wenpstevdy yklrygpmtg qevaevtvpk ssdpksrydi tglhpgteyk
  421 itvvpmrgel egkpillngr teidsptnvv tdrvtedtat vswdpvqavi dkyvvrytsa
  481 dgdtkemavh kdesstvltg lkpgeaykvy vwaergnqgs kkadtnalte idspanlvtd
  541 rvtentatis wdpvqatidk yvvrytsadd getrevlvgk eqsstvltgl rpgveytvhv
  601 waqkgdresk kadtnaptdi dspknlvtdr vtenmatvsw dpvqaaidky vvrytsagge
  661 trevpvgkeq sstvltglrp gmeymvhvwa qkgdqeskka dtkaqtdids pqnlvtdrvt
  721 enmatvswdp vratidryvv rytsakdget revpvgkeqs stvltglrpg veytvhvwaq
  781 kgaqeskkad tkaqtdidsp qnlvtdwvte ntatvswdpv qatidryvvh ytsangetre
  841 vpvgkeqsst vltglrpgme ytvhvwaqkg nqeskkadtk aqteidgpkn lvtdwvtenm
  901 atvswdpvqa tidkymvryt sadgetrevp vgkehsstvl tglrpgmeym vhvwaqkgaq
  961 eskkadtkaq teldpprnlr psavtqsggi ltwtppsaqi hgyiltyqfp dgtvkemqlg
1021 redqrfalqg leqgatypvs lvafkggrrs rnvsttlstv garfphpsdc sqvqqnsnaa
1081 sglytiylhg dasrplqvyc dmetdgggwi vfqrrntgql dffkrwrsyv egfgdpmkef
1141 wlgldklhnl ttgtparyev rvdlqtanes ayaiydffqv asskeryklt vgkyrgtagd
1201 altyhngwkf ttfdrdndia lsncalthhg gwwyknchla npngrygetk hsegvnwepw
1261 kghefsipyv elkirphgys repvlgrkkr tlrgrlrtf
Keratin, type II cytoskeletal 5 [Homo sapiens]
ACCESSION P13647
SEQ ID NO: 45
      1 msrqssysfr sggsrsfsta saitpsvsrt sftsvsrsgg gggggfgrvs lagacgvggy
    61 gsrslynlgg skrisistsg gsfrnrfgag agggygfggg agsgfgfggg agggfglggg
  121 agfgggfggp gfpvcppggi qevtvnqsll tplnlqidps iqrvrteere qiktlnnkfa
  181 sfidkvrfle qqnkvldtkw tllqeqgtkt vrqnleplfe qyinnlrrql dsivgergrl
  241 dselrnmqdl vedfknkyed einkrttaen efvmlkkdvd aaymnkvele akvdalmdei
  301 nfmkmffdae lsqmqthvsd tsvvlsmdnn rnldldsiia evkaqyeeia nrsrteaesw
  361 yqtkyeelqq tagrhgddlr ntkheisemn rmiqrlraei dnvkkqcanl qnaiadaeqr
  421 gelalkdarn klaeleealq kakqdmarll reyqelmntk laldveiaty rkllegeecr
  481 lsgegvgpvn isvvtssyss gygsgsgygg glggglgggl ggglaggssg syyssssggv
  541 glggglsvgg sgfsassgrg lgvgfgsggg ssssvkfvst tsssrksfks
periostin isoform 1 precursor [Homo sapiens]
ACCESSION NP_006466
SEQ ID NO: 46
      1 mipflpmfsl llllivnpin annhydkila hsrirgrdqg pnvcalqqil gtkkkyfstc
    61 knwykksicg qkttvlyecc pgymrmegmk gcpavlpidh vygtlgivga tttqrysdas
  121 klreeiegkg sftyfapsne awdnldsdir rglesnvnve llnalhshmi nkrmltkdlk
  181 ngmiipsmyn nlglfinhyp ngvvtvncar iihgnqiatn gvvhvidrvl tqigtsiqdf
  241 ieaeddlssf raaaitsdil ealgrdghft lfaptneafe klprgvleri mgdkvaseal
  301 mkyhilntlq csesimggav fetlegntie igcdgdsitv ngikmvnkkd ivtnngvihl
  361 idqvlipdsa kqvielagkq qttftdlvaq lglasalrpd geytllapvn nafsddtlsm
  421 dqrllklilq nhilkvkvgl nelyngqile tiggkqlrvf vyrtavcien scmekgskqg
  481 rngaihifre iikpaekslh eklkqdkrfs tflslleaad lkelltqpgd wtlfvptnda
  541 fkgmtseeke ilirdknalq niilyhltpg vfigkgfepg vtnilkttqg skiflkevnd
  601 tllvnelksk esdimttngv ihvvdkllyp adtpvgndql leilnkliky iqikfvrgst
  661 fkeipvtvyt tkiitkvvep kikviegslq piiktegptl tkvkiegepe frlikegeti
  721 tevihgepii kkytkiidgv pveiteketr eeriitgpei kytristggg eteetlkkll
  781 qeevtkvtkf ieggdghlfe deeikrllqg dtpvrklqan kkvqgsrrrl regrsq
osteocalcin preproprotein (OCN) [Homo sapiens]
ACCESSION NP_954642 NP_000702
SEQ ID NO: 47
      1 mraltllall alaalciagq agakpsgaes skgaafvskq egsevvkrpr rylyqwlgap
    61 vpypdplepr revcelnpdc deladhigfq eayrrfygpv
alkaline phosphatase [Homo sapiens]
ACCESSION AAB59378
SEQ ID NO: 48
      1 mispflvlai gtcltnslvp ekekdpkywr dqaqetlkya lelqklntnv aknvimflgd
    61 gmgvstvtaa rilkgqlhhn pgeetrlemd kfpfvalskt yntnaqvpds agtataylcg
  121 vkanegtvgv saatersrcn ttqgnevtsi lrwakdagks vgivtttrvn hatpsaayah
  181 sadrdwysdn emppealsqg ckdiayqlmh nirdidvimg ggrkymypkn ktdveyesde
  241 kargtrldgl dlvdtwksfk prykhshfiw nrtelltldp hnvdyllglf epgdmqyeln
  301 rnnvtdpsls emvvvaiqil rknpkgffll veggridhgh hegkakqalh eavemdraig
  361 qagsltssed tltvvtadhs hvftfggytp rgnsifglap mlsdtdkkpf tailygngpg
  421 ykvvggeren vsmvdyahnn yqaqsavplr hethggedva vfskgpmahl lhgvheqnyv
  481 phvmayaaci ganlghcapa ssagslaagp lllalalypl svlf
```

What is claimed is:

1. A method of treating a subject for a mineralization injury, disease or disorder comprising:
administering to a subject in need thereof a composition comprising (i) an exosome or (ii) one or more of a polypeptide, mRNA, or miRNA associated with or derived from the exosome.

2. A method of promoting dentinogenesis, amelogenesis, or odontogenesis in a subject comprising:
administering to a subject in need thereof a composition comprising (i) an exosome or (ii) one or more of a polypeptide, mRNA, or miRNA associated with or derived from the exosome.

3. The method of any one of claims 1-2, comprising contacting a dental mesenchyme cell and the composition.

4. The method of any one of claims 1-3, wherein administering results in increased expression of dentin sialophosphoprotein (DSPP) expression, increased expression of osteocalcin (OCN) expression, increased expression of alkaline phosphatase, promotion of promote calcium deposition, promotion of dentinogenesis, promotion of amelogenesis, or promotion of odontogenesis.

5. The method of any one of claims 1-4, wherein the exosome comprises an epithelium-derived exosome; mesenchyme-derived exosome; or a mesoderm-derived exosome.

6. The method of any one of claims 1-5, further comprising:
isolating the exosome from an epithelium cell, a mesenchyme cell, or a mesoderm cell.

7. The method of any one of claims 1-6, wherein
the exosome has a particle size of about 80 to about 120 nm;
a plurality of epithelium-derived exosomes have an average particle size of about 95 nm to about 105 nm; or
a plurality of mesenchyme-derived exosomes have an average particle size of about 110 nm to about 120 nm.

8. The method of any one of claims 1-7, further comprising:
isolating the exosome from a tooth epithelium cell, a tooth mesenchyme cell, or a tooth mesoderm cell.

9. The method of any one of claims 1-8, wherein the composition comprises an epithelium-derived exosome, the exosome comprising one or more of:
a miRNA comprising a nucleic acid sequence selected from the group consisting of SEQ ID NO: 1 (rno-miR-674-5p), SEQ ID NO: 2 (rno-miR-199a-3p), SEQ ID NO: 3 (rno-miR-23b-3p), SEQ ID NO: 4 (rno-miR-200b-3p£°), SEQ ID NO: 5 (rno-miR-25-3p), SEQ ID NO: 6 (rno-miR-672-5p£°), and SEQ ID NO: 7) (rno-miR-103-3p£°), or a nucleic acid sequence having at least about 90% sequence identity thereto and retaining an activity associated with the miRNA; or
a polypeptide comprising an amino acid sequence of SEQ ID NO: 34 (CTGF), SEQ ID NO: 35 (peroxiredoxin-2), SEQ ID NO: 36 (odontogenic ameloblast-associated protein precursor), SEQ ID NO: 37 (hemiferrin, transferrin-like protein), SEQ ID NO: 38 (CaBP1), SEQ ID NO: 39 (follistatin-related protein 1 precursor), and SEQ ID NO: SEQ ID NO: 40 (cofilin-1), or an amino acid sequence having at least about 90% sequence identity thereto and retaining an activity associated with the polypeptide.

10. The method of any one of claims 1-9, wherein the composition comprises one or more of:
a miRNA comprising a nucleic acid sequence selected from the group consisting of SEQ ID NO: 1 (rno-miR-674-5p), SEQ ID NO: 2 (rno-miR-199a-3p), SEQ ID NO: 3 (rno-miR-23b-3p), SEQ ID NO: 4 (rno-miR-200b-3p£°), SEQ ID NO: 5 (rno-miR-25-3p), SEQ ID NO: 6 (rno-miR-672-5p£°), and SEQ ID NO: 7) (rno-miR-103-3p£°), or a nucleic acid sequence having at least about 90% sequence identity thereto and retaining an activity associated with the miRNA;
a polypeptide comprising an amino acid sequence of SEQ ID NO: 34 (CTGF), SEQ ID NO: 35 (peroxiredoxin-2), SEQ ID NO: 36 (odontogenic ameloblast-associated protein precursor), SEQ ID NO: 37 (hemiferrin, transferrin-like protein), SEQ ID NO: 38 (CaBP1), SEQ ID NO: 39 (follistatin-related protein 1 precursor), and SEQ ID NO: SEQ ID NO: 40 (cofilin-1), or an amino acid sequence having at least about 90% sequence identity thereto and retaining an activity associated with the polypeptide; or
a vector comprising a transcribable nucleic acid molecule encoding the miRNA or the polypeptide operably linked to a promoter.

11. The method of any one of claims 9-10, wherein the composition promotes amelogenesis.

12. The method of any one of claims 1-11, wherein the composition comprises an mesenchyme-derived exosome, the exosome comprising one or more of:
a miRNA comprising a nucleic acid sequence selected from the group consisting of SEQ ID NO: 8 (rno-let-7c-5p£°), SEQ ID NO: 9 (rno-let-7a-5p£°), SEQ ID NO: 10 (rno-let-7d-5p£°), SEQ ID NO: 11 (rno-miR-352£°), SEQ ID NO: 12 (rno-miR-532-3p£°), SEQ ID NO: 13 (rno-miR-181b-5p£°), SEQ ID NO: 14) (rno-miR-23b-3p£°), SEQ ID NO: 15 (rno-miR-93-5p£°), SEQ ID NO: 16 (rno-miR-16-5p£°), SEQ ID NO: 17 (rno-miR-103-3p£°), SEQ ID NO: 18 (rno-miR-151-5p£°), and SEQ ID NO: 19 (rno-miR-99b-5p£°), or a nucleic acid sequence having at least about 90% sequence identity thereto and retaining an activity associated with the miRNA; or
a polypeptide comprising an amino acid sequence of SEQ ID NO: 41 (annexin II), SEQ ID NO: 42 (lactadherin isoform b precursor), SEQ ID NO: 43 (pigment epithelium-derived factor precursor), SEQ ID NO: 44 (tenascin-N precursor), SEQ ID NO: 45 (keratin, type II cytoskeletal 5), and SEQ ID NO: 46 (periostin isoform 1 precursor), or an amino acid sequence having at least about 90% sequence identity thereto and retaining an activity associated with the polypeptide.

13. The method of any one of claims 1-12, wherein the composition comprises one or more of:
a miRNA comprising a nucleic acid sequence selected from the group consisting of SEQ ID NO: 8 (rno-let-7c-5p£°), SEQ ID NO: 9 (rno-let-7a-5p£°), SEQ ID NO: 10 (rno-let-7d-5p£°), SEQ ID NO: 11 (rno-miR-352£°), SEQ ID NO: 12 (rno-miR-532-3p£°), SEQ ID NO: 13 (rno-miR-181b-5p£°), SEQ ID NO: 14) (rno-miR-23b-3p£°), SEQ ID NO: 15 (rno-miR-93-5p£°), SEQ ID NO: 16 (rno-miR-16-5p£°), SEQ ID NO: 17 (rno-miR-103-3p£°), SEQ ID NO: 18 (rno-miR-151-5p£°), and SEQ ID NO: 19 (rno-miR-99b-5p£°), or a nucleic acid sequence having at least about 90% sequence identity thereto and retaining an activity associated with the miRNA;
a polypeptide comprising an amino acid sequence of SEQ ID NO: 41 (annexin II), SEQ ID NO: 42 (lactadherin isoform b precursor), SEQ ID NO: 43 (pigment epithelium-derived factor precursor), SEQ ID NO: 44 (tenascin-N precursor), SEQ ID NO: 45 (keratin, type II cytoskeletal 5), and SEQ ID NO: 46 (periostin isoform 1 precursor), or an amino acid sequence having at least about 90% sequence identity thereto and retaining an activity associated with the polypeptide; or
a vector comprising a transcribable nucleic acid molecule encoding the miRNA or the polypeptide operably linked to a promoter.

14. The method of any one of claims 12-13, wherein the composition promotes odontogenesis.

15. The method of any one of claims 1-14, wherein the exosome comprises one or more of:
a miRNA comprising a nucleic acid sequence selected from the group consisting of SEQ ID NO: 20 (rno-miR-135b-5p£°), SEQ ID NO: 21 (rno-miR-200a-3p£°), SEQ ID NO: 22 (rno-miR-200b-3p£°), SEQ ID NO: 23 (rno-miR-200b-5p£°), SEQ ID NO: 24 (rno-miR-200c-3p£°), SEQ ID NO: 25 (rno-miR-21-3p£°), SEQ ID NO: 26 (rno-miR-21-3p£°), SEQ ID NO: 27 (rno-miR-15b-3p£°), SEQ ID NO: 28 (rno-miR-15b-5p£°), SEQ ID NO: 29 (rno-miR-16-5p£°), SEQ ID NO: 30 (rno-miR-122-5p£°), SEQ ID NO: 31 (rno-miR-203a-3p£°), and SEQ ID NO: 32 (rno-miR-375-3p£°), or a nucleic acid sequence having at least about 90% sequence identity thereto and retaining an activity associated with the miRNA.

16. The method of any one of claims 1-15, wherein the composition comprises:
one or more of a miRNA comprising a nucleic acid sequence selected from the group consisting of SEQ ID NO: 20 (rno-miR-135b-5p£°), SEQ ID NO: 21 (rno-miR-200a-3p£°), SEQ ID NO: 22 (rno-miR-200b-3p£°), SEQ ID NO: 23) (rno-miR-200b-5p£°), SEQ ID NO: 24 (rno-miR-200c-3p£°), SEQ ID NO: 25 (rno-miR-21-3p£°), SEQ ID NO: 26 (rno-miR-21-3p£°), SEQ ID NO: 27 (rno-miR-15b-3p£°), SEQ ID NO: 28 (rno-miR-15b-5p£°), SEQ ID NO: 29 (rno-miR-16-5p£°), SEQ ID NO: 30 (rno-miR-122-5p£°), SEQ ID NO: 31 (rno-miR-203a-3p£°), and SEQ ID NO: 32 (rno-miR-375-3p£°), or a nucleic acid sequence having at least about 90% sequence identity thereto and retaining an activity associated with the miRNA; or
a vector comprising a transcribable nucleic acid molecule encoding the miRNA operably linked to a promoter.

17. The method of any one of claims 1-16, wherein the subject is a mammal.

18. The method of any one of claims 1-17, wherein the subject is a human.

19. The method of any one of claims 1-18, wherein the mineralization injury, disease or disorder is selected from the group consisting of bone fracture, tooth extraction sockets, periodontal defects, non-unions, dental and orthopedic implant integration, and bony augmentation in reconstructive or plastic procedures.

20. A composition for treating a mineralization injury, disease or disorder or for promoting dentinogenesis, amelogenesis, or odontogenesis, the composition comprising:
(a) an epithelium-derived exosome, a mesenchyme-derived exosome, or a mesoderm-derived exosome; and
(b) one or more of the following:
(i) a miRNA comprising a nucleic acid sequence selected from the group consisting of SEQ ID NO: 1 (rno-miR-674-5p), SEQ ID NO: 2 (rno-miR-199a-3p), SEQ ID NO: 3 (rno-miR-23b-3p), SEQ ID NO: 4 (rno-miR-200b-3p£°), SEQ ID NO: 5 (rno-miR-25-3p), SEQ ID NO: 6 (rno-miR-672-5p£°), and SEQ ID NO: 7 (rno-miR-103-3p£°), or a nucleic acid sequence having at least about 90% sequence identity thereto and retaining an activity associated with the miRNA;
(ii) a polypeptide comprising an amino acid sequence of SEQ ID NO: 34 (CTGF), SEQ ID NO: 35 (peroxiredoxin-2), SEQ ID NO: 36 (odontogenic ameloblast-associated protein precursor), SEQ ID NO: 37 (hemiferrin, transferrin-like protein), SEQ ID NO: 38 (CaBP1), SEQ ID NO: 39 (follistatin-related protein 1 precursor), and SEQ ID NO: SEQ ID NO: 40 (cofilin-1), or an amino acid sequence having at least about 90% sequence identity thereto and retaining an activity associated with the polypeptide;
(iii) a miRNA comprising a nucleic acid sequence selected from the group consisting of SEQ ID NO: 1 (rno-miR-674-5p), SEQ ID NO: 2 (rno-miR-199a-3p), SEQ ID NO: 3 (rno-miR-23b-3p), SEQ ID NO: 4 (rno-miR-200b-3p£°), SEQ ID NO: 5 (rno-miR-25-3p), SEQ ID NO: 6 (rno-miR-672-5p£°), and SEQ ID NO: 7 (rno-miR-103-3p£°), or a nucleic acid sequence having at least about 90% sequence identity thereto and retaining an activity associated with the miRNA;
(iv) a polypeptide comprising an amino acid sequence of SEQ ID NO: 34 (CTGF), SEQ ID NO: 35 (peroxiredoxin-2), SEQ ID NO: 36 (odontogenic ameloblast-associated protein precursor), SEQ ID NO: 37 (hemiferrin, transferrin-like protein), SEQ ID NO: 38 (CaBP1), SEQ ID NO: 39 (follistatin-related protein 1 precursor), and SEQ ID NO: SEQ ID NO: 40 (cofilin-1), or an amino acid sequence having at least about 90% sequence identity thereto and retaining an activity associated with the polypeptide; or
(v) a vector comprising a transcribable nucleic acid molecule encoding the miRNA or the polypeptide operably linked to a promoter; wherein the (b) is independently present in the composition or contained within the exosome.
